(19) United States
(12) Patent Application Publication
(10) Pub. No.: US 2026/0256652 A1
(43) Pub. Date: Sep. 3, 2026

(54) MOTORIZED SYRINGE ASSISTANCE DEVICE AND METHOD OF USE THEREOF

(71) Applicant: United Therapeutics Corporation, Silver Spring, MD (US)

(72) Inventors: Avi Azoulay, Silver Spring, MD (US); Adam James Elias, Silver Spring, MD (US); Ian Voos, Sivler Spring, MD (US); Assaf Shaked, Silver Spring, MD (US); Thomas Baran, Silver Spring, MD (US); Lauren Morgan-Schatz, Silver Spring, MD (US); Timothy Atolagbe, Silver Spring, MD (US); Samuel A. Mancuso, Silver Spring, MD (US)

(73) Assignee: United Therapeutics Corporation, Silver Spring, MD (US)

(21) Appl. No.: 19/552,292

(22) Filed: Feb. 27, 2026

Related U.S. Application Data
(60) Provisional application No. 63/764,941, filed on Feb. 28, 2025.

(51) Int. Cl. A61J 1/20; A61J 1/22

(52) U.S. Cl. CPC A61J 1/2055; A61J 1/2065; A61J 1/22

(57) ABSTRACT

A device for administering a medicine includes a frame, and an actuator system coupled to the frame. The actuator system includes a motor configured to drive the actuator from a first position to a second position in a first direction, a gripper coupled to a first end of the actuator and configured to receive a barrel of a syringe, and a plunger holder coupled to the actuator system and configured to receive a plunger of a syringe. The gripper is configured to grip the barrel of the syringe in a stationary position and the actuator system is configured to move the plunger of the syringe from the first position to the second position.

### CROSS-REFERENCE TO RELATED PATENT APPLICATION

This application claims the benefit of and priority to U.S. Provisional Patent Application No. 63/764,941, filed Feb. 28, 2025, which is incorporated herein by reference in its entirety.

### BACKGROUND

The present disclosure relates generally to a device for providing mechanical assistance in filling a syringe with a desired amount of liquid and expelling the liquid into a container, for example for mixing the liquid with another liquid. The device assists a user in drawing a liquid (such as a diluent) into a syringe, moving the liquid to a different container (e.g., a cassette), and/or mixing the diluent with a medication (e.g., in the cassette). More specifically, the present disclosure relates to medical devices that may be utilized in an at-home care setting to mechanically assist a user with filling syringes and transferring the fluid in the filled syringes into a second container, such as a cassette or drug delivery device.

Syringes configured to receive a large amount of fluid, such as 50 mL syringes, have a significant amount of resistance requiring a large amount of force to draw a fluid into the syringe. Repeatedly drawing large amount of fluid into the syringe and pressing the fluid out of the syringe for mixing can cause fatigue in the patient and/or caregiver, and/or may be beyond the physical capabilities of some individuals.

Various medications are provided as a concentrated solution and are intended for mixing with a diluent. The mixing, or diluting process, can occur in an at home setting which reduces the burden of having to visit a healthcare provider or pharmacy to have the medication mixed with diluent. It is further advantageous to reduce the burden on pharmacies by having the patient mix medication with diluent themselves. In some instances, for example due to stability of the medication, the medication must be diluted or mixed within a certain time period prior to administration. Fluid medications and buffer solutions and/or diluents are often stored in vials in which a syringe is used to extract the fluids. The dilutant and in some cases, the medication, must be extracted via a syringe and mixed. It is often imperative that each fluid is precisely measured.

Extracting the fluids with a syringe requires fine motor skills and a considerable amount of strength, especially when multiple vials need to be extracted and or mixed requiring the patient to both extract the fluids into the syringe and expel the fluids out of the syringe multiple times. Larger syringes, such as 50 mL syringes, with large pistons or plungers and long barrels require a large amount of force to draw the diluent into the syringe making the mixing process difficult and tiresome especially when multiple large syringes need to be filled and mixed with a medication. The patient may further need to extract additional fluid or expel excess fluid from each syringe in small amounts, such as 1-2 mL, which adds an additional layer of difficulty to patients with weakened dexterity.

This process can be quite time consuming and difficult for patients but necessary to receive life-saving medications. This often results in the need to hire an in-home caregiver multiple times a week to assist in filing syringes and preparing the medication for delivery which can be a burden to the patient.

### SUMMARY

At least one embodiment relates to a device for filling a syringe and/or using a syringe to transfer fluid between a first container and a second container. The device includes a frame, a gripper coupled to the frame and configured to grip a barrel of the syringe, and an actuator system coupled to the frame. The actuator system includes a motor and a plunger holder configured to receive a plunger of the syringe when the gripper grips the barrel of the syringe. The motor is configured to drive the plunger holder between at least a first position, a second position, and a third position. While the device can be used for any diluent or medicine that is supplied in a vial as a concentrated solution that needs to be diluted to form a solution at the required dosage for administration to the patient, the device preferably is configured to operate with a vial comprising a diluent or a medication, such as treprostinil or a pharmaceutically acceptable salt or ester thereof.

Another embodiment relates to a system. The system includes a syringe including a barrel and a plunger extending from a first end of the barrel, and a container comprising a liquid. The container is selectively fluidly coupled to the syringe. The system also includes a device including a frame, a gripper coupled to the frame and configured to receive the barrel of the syringe in a vertical orientation, and an actuator system. The actuator system includes an actuator plate coupled to the frame, a plunger holder configured to receive the plunger of the syringe when the gripper grips the barrel of the syringe, and a motor coupled to the actuator plate and configured to drive the plunger holder between at least a first position, a second position, and a third position.

Another embodiment relates to a method of operating a device. The method includes in response to a first input received by a first button of the device, moving a plunger of a syringe from a first position to a second position drawing air into a barrel of the syringe, in response to a second input received by the first button, moving the plunger from the second position to the third position pushing air into a vial, moving the plunger from the third position to the second position drawing a fluid from the vial into the barrel of the syringe, and in response to a third input to a second button of the device, moving the plunger from the second position to the first position pushing the fluid into a cassette.

This summary is illustrative only and is not intended to be in any way limiting. Other aspects, inventive features, and advantages of the devices or processes described herein will become apparent in the detailed description set forth herein, taken in conjunction with the accompanying figures, wherein like reference numerals refer to like elements.

### BRIEF DESCRIPTION OF THE FIGURES

The disclosure will become more fully understood from the following detailed description, taken in conjunction with the accompanying figures, wherein like reference numerals refer to like elements, in which:

FIG. 1 is a front perspective of a device for transferring fluid using a syringe, according to an exemplary embodiment.

FIG. 2 is a back perspective view of the device for transferring fluid using a syringe, according to the embodiment of FIG. 1.

FIG. 3 is a perspective cutaway view of a portion of the device, according to the embodiment of FIG. 1.

FIG. 4 is a perspective view of an interior portion of the device, according to the embodiment of FIG. 1.

FIG. 5 is a front perspective cutaway view of a portion of the device, according to the embodiment of FIG. 1.

FIG. 6A is a front perspective view of a device for transferring fluid using a syringe, according to another exemplary embodiment.

FIG. 6B is a back perspective view of the device according to the embodiment of FIG. 6A.

FIG. 7A is a front view the device with a syringe in a first position, according to the embodiment of FIG. 1.

FIG. 7B is a front view the device with the syringe in the first position transitioning to a second position, according to the embodiment of FIG. 7A.

FIG. 7C is a front view the device with the syringe in the second position and coupled to a container of diluent, according to the embodiment of FIG. 7A.

FIG. 8A is a front view the device with the syringe in the second position and coupled to the container of diluent, according to the embodiment of FIG. 1.

FIG. 8B is a front view the device with the syringe transition from the second position to the first position and coupled to the container of diluent, according to the embodiment of FIG. 1.

FIG. 8C is a front view the device with the syringe in the second position, according to the embodiment of FIG. 1.

FIG. 9A is a front view the device with the syringe in the second position, according to the embodiment of FIG. 1.

FIG. 9B is another front view the device with the syringe in the second position, according to the embodiment of FIG. 1.

FIG. 9C is a front view the device with the syringe in the first position, according to the embodiment of FIG. 1.

FIG. 10 is a flowchart of a method of using the device of FIG. 1, according to an exemplary embodiment.

FIG. 11 is a perspective view of a kit including the device of FIG. 1, according to an exemplary embodiment.

FIG. 12 is another perspective view of the kit of FIG. 11.

FIG. 13 a perspective view of a portion of the kit of FIG. 11, according to an exemplary embodiment.

FIG. 14 is a front perspective view of a device for transferring fluid using a syringe, according to another exemplary embodiment.

FIG. 15 is a front perspective view of the device of FIG. 14.

FIG. 16 is an expanded view of the device of FIG. 14.

FIG. 17 is an expanded view of a portion of the device of FIG. 14.

FIG. 18 is a side view of a portion of the device of FIG. 14.

FIG. 19 is a front view of a user interface of the device of FIG. 14.

### DETAILED DESCRIPTION

Before turning to the figures, which illustrate certain exemplary embodiments in detail, it should be understood that the present disclosure is not limited to the details or methodology set forth in the description or illustrated in the figures. It should also be understood that the terminology used herein is for the purpose of description only and should not be regarded as limiting.

Referring generally to the figures, the various exemplary embodiments disclosed herein relate to systems, apparatuses, and method for providing mechanical assistance for extracting a fluid, such as a diluent, from a first container, such as a vial, and transferring, or expelling, the fluid into a second container, such as a cassette. One goal of the teachings herein is for each fluid, such as a diluent and a medication to be precisely measured and mixed in preparation for administering the diluted medication to the patient. Preferably, the diluent is a glycine diluent, sterile water, or sodium chloride diluent, and preferably the medication is Remodulin® (treprostinil solution for injection, supplied in 20-mL vials containing 2 mg (0.1 mg/mL), 4 mg (0.2 mg/mL), 8 mg (0.4 mg/mL), 20 mg (1 mg/mL), 50 mg (2.5 mg/mL), 100 mg (5 mg/mL), 200 mg (10 mg/mL), or 400 mg (20 mg/mL) of treprostinil). Remodulin® is a prostacyclin mimetic indicated for:
Treatment of pulmonary arterial hypertension (PAH; WHO Group 1) to diminish symptoms associated with exercise. Studies establishing effectiveness included patients with NYHA Functional Class II-IV symptoms and etiologies of idiopathic or heritable PAH (58%), PAH associated with congenital systemic-to-pulmonary shunts (23%), or PAH associated with connective tissue diseases (19%).
Patients who require transition from epoprostenol, to reduce the rate of clinical deterioration.

Remodulin® is administered as follows in PAH WHO Group 1 patients with NYHA Class II-IV symptoms:
Initial dose for patients new to prostacyclin infusion therapy: 1.25 ng/kg/min; increase based on clinical response (increments of 1.25 ng/kg/min per week for the first 4 weeks of treatment, later 2.5 ng/kg/min per week).
Mild to moderate hepatic insufficiency: Decrease initial dose to 0.625 ng/kg/min.
   Remodulin® can be administered with or without further dilution with Sterile Diluent for Remodulin or similar approved high-pH glycine diluent (e.g., Sterile Diluent for Flolan or Sterile Diluent for Epoprostenol), Sterile Water for Injection, or 0.9% Sodium Chloride Injection prior to administration. See Table 1 and Section 16 of the Remodulin® FDA approved label reproduced below for storage and administration time limits for the different diluents. Diluted Remodulin® has been shown to be stable at ambient temperature when stored for up to 14 days using high-pH glycine diluent at concentrations as low as 0.004 mg/mL (4,000 ng/mL).

TABLE 1
Selection of Diluent

| Diluent | Storage Limits | Administration Limits |
|---|---|---|
| None | See Section 16 | 16 weeks at 40° C. |
| Sterile Diluents for | 14 days at room | 48 hours at 40° C. |
| Remodulin, Flolan, | temperature | |
| or Epoprostenol | | |
| Sterile Water for | 4 hours at room | 48 hours at 40° C. |
| Injection 0.9% Sodium | temperature or | |
| Chloride for Injection | 24 hours refrigerated | |

Remodulin is supplied in 20-mL multidose vials as sterile solutions in water for injection, individually packaged in cartons. Unopened vials of Remodulin are stable until the date indicated when stored at 25° C. (77° F.), with excursions permitted to 2-30° C. (36-86° F.). A single vial of Remodulin should be used for no more than 30 days after the initial introduction into the vial. Remodulin Injection is supplied as:

| | | |
|---|---|---|
| Remodulin | Concentration | NDC |

| | | | |
|---|---|---|---|
| 2 mg/20 mL | 0.1 | mg/mL | 66302-111-01 |
| 4 mg/20 mL | 0.2 | mg/mL | 66302-112-01 |
| 8 mg/20 mL | 0.4 | mg/mL | 66302-114-01 |
| 20 mg/20 mL | 1 | mg/mL | 66302-101-01 |
| 50 mg/20 mL | 2.5 | mg/mL | 66302-102-01 |
| 100 mg/20 mL | 5 | mg/mL | 66302-105-01 |
| 200 mg/20 mL | 10 | mg/mL | 66302-110-01 |
| 400 mg/20 mL | 20 | mg/mL | 66302-120-01 |

Sterile Diluent for Remodulin is supplied separately as: 50-mL vial, carton of 1 (NDC 66302-150-50).

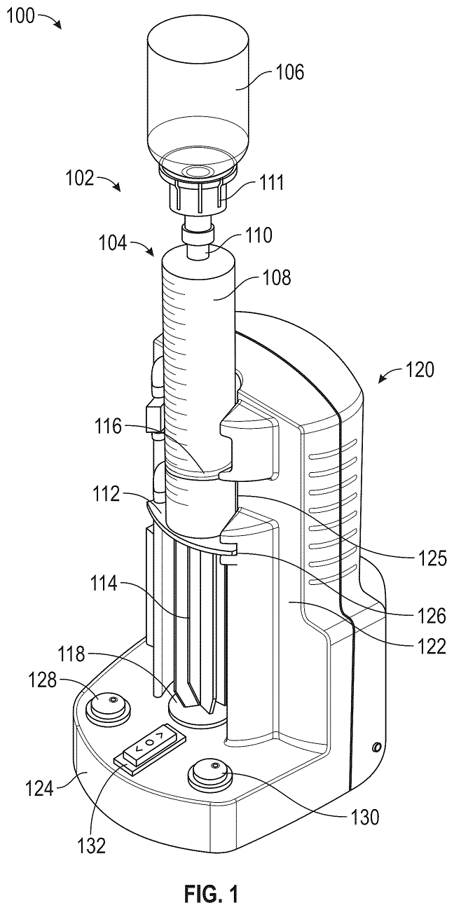

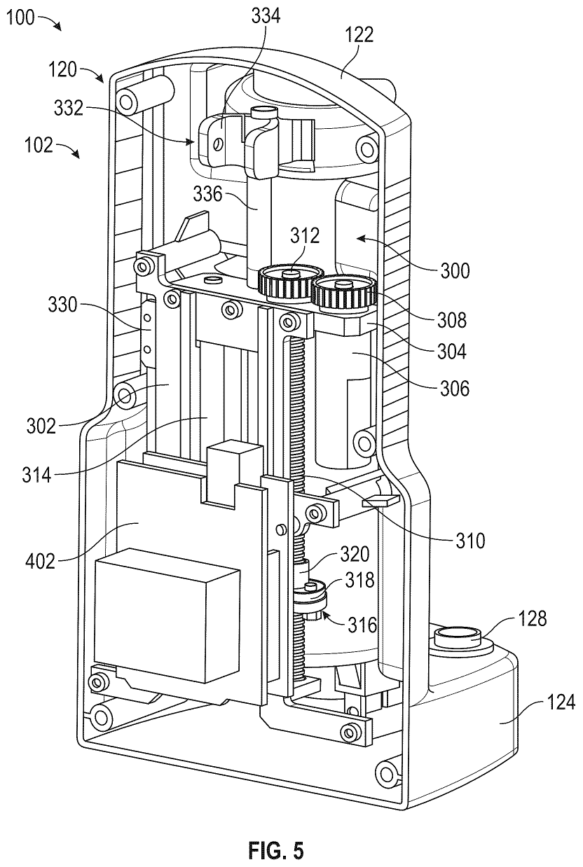

As shown in FIGS. 1-8C, the present disclosure relates to a system 100 for filling a syringe with a liquid, such as a diluent, and providing the liquid into a cassette or other device for mixing fluids, such as diluents, buffers, and medications in preparation for delivery. The system includes a device 102, a syringe 104, and a first fluid container, such as a vial 106.

As shown in FIG. 1, the syringe 104 includes syringe barrel 108. The syringe barrel 108 is a cylindrical body configured to receive a fluid, such as a fluid medication or a diluent. The syringe barrel 108 includes markings including hashes and numbering for measuring an amount of fluid drawn into the syringe barrel 108.

The syringe 104 also includes a first end, such as a Luer lock or a nozzle 110 and a body flange 112. The body flange 112 is positioned on a distal end of the syringe barrel 108 opposite the nozzle 110. The nozzle 110 includes a coupling mechanism configured to receive and/or couple to the vial 106.

As shown in FIG. 1, the system 100 includes an adapter 111. The adapter 111 is coupled to the syringe 104 and configured to couple the vial 106 to the syringe 104. For example, a first end of the adapter 111 is coupled, for example, screwed or pressed onto, the nozzle 110 and a second end of the adapter 111 is coupled to the vial 106. The adapter 111 is configured to secure the vial 106 to the syringe 104.

The syringe 104 also includes a plunger 114. The plunger 114 includes a stopper end 116 and a plunger base 118. The stopper end 116 is positioned within the syringe barrel 108. The plunger base 118 is positioned on an end of the plunger 114 opposite the stopper end 116.

The device 102 includes a casing 120. According to this embodiment, the casing 120 is manufactured of or comprises a plastic material. The casing 120 includes a body portion 122 and a base portion 124. The base portion 124 extends outward substantially perpendicular to the body portion 122.

The body portion 122 defines a cylindrical cavity 125 configured to receive the syringe 104. The cylindrical cavity 125 extends vertically along the body portion 122 of the casing 120. The cylindrical cavity 125 is sized to receive various sizes of syringes 104 (e.g., 10 mL, 20 mL, 50 mL, etc.). However, according to this embodiment, the cylindrical cavity 125 is sized specifically to receive a 50 mL syringe.

The body portion 122 also defines a body receptacle 126. The body receptacle 126 is configured to receive the body flange 112. As shown in FIG. 1, the body receptacle is configured to couple the body flange 112 to the casing 120 such that the body flange 112 and the syringe barrel 108 are held in a stationary position.

As described in more detail below, the syringe barrel 108 is received by, and selectively coupled to, the casing 120 of the device 102. When coupled to, or secured to, the casing 120, the syringe barrel 108 is vertically oriented with respect to the body portion 122 (e.g., extends parallel to the device 102, etc.). By positioning the syringe barrel 108 in a vertical orientation, air (e.g., air bubbles, etc.) that may enter the syringe barrel 108 will float to a top end of the syringe barrel 108 (e.g., an end opposite the plunger 114, an end near the vial 106, etc.), such that the air bubbles can be easily expelled or forced out of the syringe barrel 108 once filled with the desired amount of fluid. It is advantageous to minimize air bubbles within the syringe barrel 108 to minimize the transmission of air into another device or container, such as a cassette.

As shown in FIG. 1, the vial 106 is also vertically oriented. According to this embodiment, the vial 106 is flipped upside down, such that a vial port of the vial 106 is adjacent to, or nearest, the syringe 104. As previously described, the vial 106 is coupled to the syringe 104 via the adapter 111. For example, the vial port end of the vial 106 may be press-fit into the adapter 111 and then the adapter is coupled to, or secured to, the syringe 104. As such, the fluid within the vial 106 flows down towards the vial port and air, and air pressed into the vial (e.g., to create pressure within the vial, and minimize a vacuum, etc.) flows towards an end opposite the vial port. Vertically aligning the syringe 104 and the vial 106 further minimizes the transmission of air bubble back into the syringe barrel 108 when drawing fluid into the syringe barrel 108.

The base portion 124 includes a plurality of buttons 128, 130, 132. As described in further detail below, the buttons 128, 130, 132 enable user control of the device 102 for moving the plunger 114 relative to the syringe barrel 108 for filling the syringe barrel 108 with fluid and/or discharging fluid (and/or air) from the syringe barrel 108.

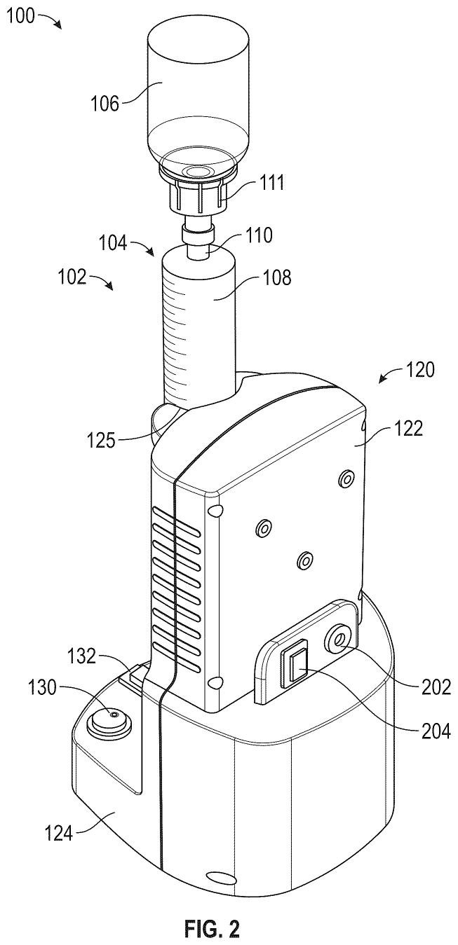

Now referring to FIG. 2 is a back view of the system 100. The casing 120 includes a power port 202 and a power button or switch 204. According to this embodiment, the device 102 is an electric device configured to receive power from an external power source. For example, the power port 202 is configured to couple to a cord that electrically couples the device 102 to an outlet. The power switch 204 is configured to, when engage, activate the device 102 for use. In other embodiments, the device 102 is battery-powered.

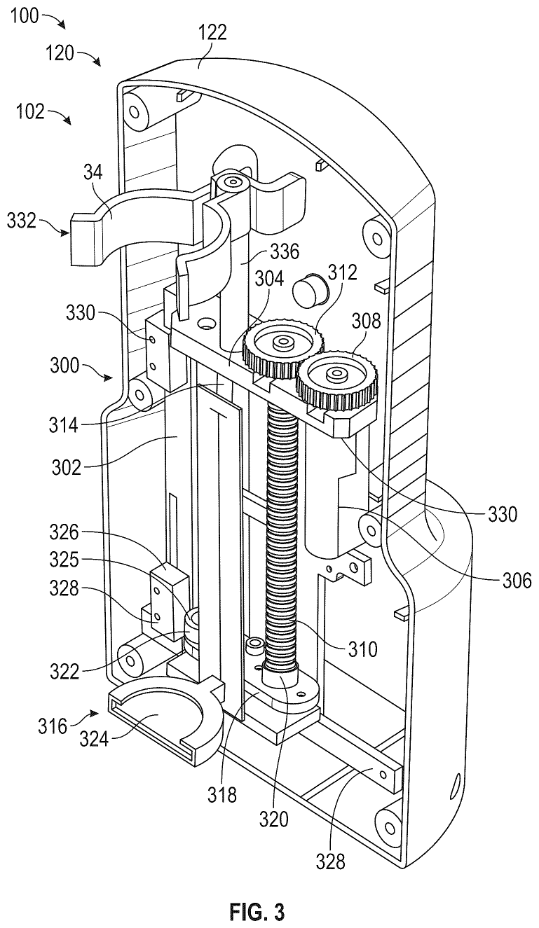

Now referring to FIG. 3-5, is an interior view of a portion of the device 102 as shown in FIGS. 1 and 2. As shown in FIG. 3, the device 102 includes an actuator system 300. The actuator system 300. The actuator system 300 is configured to drive the plunger 114 of the syringe 104 in a first direction (e.g., downward, etc.) drawing or pulling fluid into the syringe barrel 108 and drive the plunger 114 of the syringe 104 in a second direction (e.g., upward, etc.) expelling or releasing air and/or fluid from the syringe barrel 108. In particular, as will be described in further detail below, the actuator system 300 includes a plunger holder 316 and a motor 306, where the plunger holder 316 holds the plunger base 118 and is driven by operation of the motor 306 to draw fluid in or force fluid out of the syringe 104.

The actuator system 300 includes a frame 302. The frame 302 is configured to couple and/or position the actuating system within the casing 120. According to the embodiment the frame 302 comprises a metal, or a hardened or rigid plastic.

The actuator system 300 includes an actuator plate 304, a motor 306, and a first gear 308. The actuator plate 304 is coupled to the frame 302. A portion of the motor 306 is positioned through the actuator plate 304 and coupled to the first gear 308 coupling the motor 306 to the actuator plate 304 at a first end of the frame (e.g., an end opposite from the base portion 124 of the casing 120, etc.). The motor 306 is configured to rotatably drive the first gear 308 in a first direction (e.g., a clockwise direction, etc.) and a second direction (e.g., opposite the first direction, a counter-clockwise direction, etc.).

The actuator system 300 further includes a first rod, such as a threaded rod, threaded screw, or a lead screw, 310, a second gear 312, and a second rod, or a guide rod 314. Threaded screw 310 is positioned adjacent to the motor 306 (e.g., proximal to a center of the device 102, etc.) and extends from the actuator plate 304 to a base of the frame 302 (e.g., a portion of the frame adjacent to the base portion 124 of the casing 120, etc.). A portion of threaded screw 310 extends through the actuator plate 304 and is coupled to the second gear 312 rotatably coupling or securing threaded screw 310 to the actuator plate 304. The threaded screw 310 is configured to rotate within the casing 120. The motor 306 drives the rotation of the first gear 308, and the first gear 308 engages the second gear 312 causing the rotation of the threaded screw 310. The rotation of the threaded screw 310 moves the plunger 114 of the syringe 104 in the first direction and the second direction.

The guide rod 314 is positioned on an end of the actuator plate 304 opposite an end where the motor 306 is positioned. The guide rod 314 is coupled to each of the actuator plate 304 and the frame 302. The guide rod 314 extends from the actuator plate 304 to the base of the frame 302. The guide rod 314 is configured to support the actuator plate 304 and facilitate the movement of the plunger 114. For example, the guide rod 314 provides linear guidance of the plunger holder 316 parallel to a vertical axis defined by the guide rod 314.

As shown in FIGS. 3-5, the actuator system 300 includes a plunger holder 316 which is coupled to the guide rod 314 and the threaded shaft 310. In particular, the plunger holder 316 includes a plate 318, which includes and extends between a first ring 320 which receives the threaded shaft 310 and a second ring 322 that receives the guide rod 314. The plunger holder 316 also includes a slotted platform 324 extending from the holder plate 318 such that the slotted platform 324 extends substantially perpendicular to the vertical axis defined by the guide rod 314 and the threaded shaft 310. The first ring 320 is a lead nut or a threaded nut, which receives the threaded screw 310, and the second ring 322 may be an internally-smooth cylinder that receives the guide rod 314. According to this embodiment, the first ring 320 is threaded and the second ring 322 is smooth, or not threaded. The plunger holder 316 is rotatably coupled to the threaded screw 310 via the first ring 320 and slidably coupled to the guide rod 314 via the second ring 322.

The slotted platform 324 is substantially semi-circular in shape. The slotted platform 324 is configured to receive the plunger base 118 releasably coupling the plunger base 118 to the actuator system 300. For example, the slotted platform 324 receives the plunger base 118 and couples the plunger base 118 to the actuator system 300 such that the actuator system 300 can selectively drive the plunger 114 in the first direction and the second direction. According to this embodiment, the plunger base 118 is configured to slide into the slotted platform 324.

The motor 306 is configured to drive the first gear 308 that engages with the second gear 312 rotating threaded screw 310. The rotation of threaded screw 310 engaging with the first ring 320 causes the holder plate 318 including the slotted platform 324 to move in the first direction or the second direction along the threaded screw 310. The second ring 322 slides along the guide rod 314, guiding the holder plate 318 in vertical movement along each of the threaded screw 310 and guide rod 314.

The holder plate 318 further includes a protrusion 325 extending from the second ring 322. The protrusion 325 is substantially pointed or triangular in shape and configured to engage with a marking to determine a position of the plunger 114. For example, the protrusion 325 extends outward from a side of the second ring 322 substantially perpendicular to the guide rod 314. As the holder plate 318 of the plunger holder 316 moves along the threaded screw 310 and guide rod 314, the protrusion 325 engages with a marking, or a sensor, that indicates a positioned of the plunger holder 316 and communicates to the actuator system 300 to stop moving or driving the plunger 114 of the syringe 104.

The device 102 also includes a plurality of indicators, electrical markers, switches, buttons, or sensors, such as limit markers 326, 328, 330. The limit markers 326, 328, 330 are electrically coupled to or communicably coupled to the circuit board 402 which uses signals from the limit markers 326, 328, 330 to control the motor 306. As such, the limit markers 326, 328, 330 are configured to engage with or make contact with the protrusion 325. For example, as the first ring 320 engages with the threaded screw 310 causing the second ring 322 to move along the guide rod 314 and the holder plate 318 of the plunger holder 316 to move vertically along each of the threaded screw 310 and the guide rod 314, the protrusion 325 protrudes laterally outward from the second ring 322 and contacts one of the limit markers 326, 328, 330.

When the protrusion 325 contacts one of the limit markers 326, 328, 330, the corresponding limit marker 326, 328, or 330 provides a signal (e.g., an electrical signal or impulse, etc.) to the circuit board 402. The circuit board 402 then sends an electrical signal or command to the motor 306 to control operation of the motor 306. For example, the electrical signal sent from the circuit board 402 to the motor 306 may command the motor 306 to stop moving or rotating the first gear 308 stopping the movement of the plunger holder 316 or command the motor 306 to reverse the rotational direction of the first gear 308 causing the plunger holder 316 to move in an opposite direction. According to this embodiment, the limit marker 330 corresponds to the first position, the limit marker 328 corresponds to the second position, and the limit marker 326 corresponds to the third position. The limit marker 326 is positioned between the limit marker 330 corresponding to the first position and the limit marker 328 corresponding to the second position. The circuit board 402 controls the motor 306 to cause movement of the plunger holder 316 between a position where the protrusion 325 engages with the limit switch 328 and a position where protrusion 325 engages limit switch 326. In response to engagement of the limit switch 328, the circuit board 402 stops operation of the motor 306 such that the plunger holder 316 stops moving in the first direction (e.g., downward, etc.). Similarly, in response to engagement of the limit switch 326, the circuit board 402 stops operation of the motor 306 such that the plunger holder 316 stops moving in the second direction (e.g., upward, etc.).

In response to a depression of multiple buttons, such as the first button 128 and the second button 130, the circuit board 402 is configured to control the motor 306 to cause the plunger holder 316 to move in the first direction downward towards the limit marker 328. In response, the protrusion 325 engaging with the limit marker 328, the limit marker 328 sends a signal to the circuit board circuit board 402. In response to receiving the signal, the circuit board 402 controls the motor 306 to stop rotating the first gear 308 stopping the movement of the plunger holder 316. In response to a depression of a button, such as the first button 128, the circuit board 402 causes the motor 306 of the actuator system 300 to rotate the first gear 308 causing the plunger 114 to move (e.g., move upward, etc.) until the protrusion 325 engages with limit marker 326. When the protrusion 325 engages with the limit marker 326, the limit marker 326 communicates the engagement with the protrusion 325 to the circuit board 402. In response to receiving such a signal from the limit marker 326, the circuit board 402 causes the motor 306 to stop rotating the 308 in the second direction (e.g., stop moving the plunger holder 316 upward, etc.) and cause the motor 306 to rotate the first gear 308 in an opposite direction moving the plunger holder 316 in an opposite direction (e.g., downward) towards the limit marker 328 (e.g., switch from moving upward to moving downward, etc.).

In some embodiments, the limit markers 326, 328, 330 are configured to communicate with the circuit board 402 such that in response to engaging with one of the limit markers 326, 328, 330, the circuit board 402 automatically controls the motor 306 to rotate the first gear 308 in an opposite direction causing the plunger holder 316 to move in an opposite direction. For example, the circuit board 402 controls the motor 306 to move the plunger holder 316 upward towards the limit marker 326, and in response to the protrusion 325 engaging with the limit marker 326 and circuit board 402 receiving a signal from the limit marker 326, the circuit board 402 controls the motor 306 to move the first gear 308 in an opposite direction causing the plunger holder 316 to move downward towards the limit marker 328. Once the protrusion 325 engages with the limit marker 328 and the circuit board 402 receives a signal from the limit marker 328, the circuit board 402 control the motor 306 to move the 308 in the opposite direction causing the plunger holder 316 to move back upward towards the limit marker 326. The circuit board 402 controls the motor 306 to repeatedly cause the plunger holder 316 move between the limit marker 326 and the limit marker 328 to an amount of push air into vial 106 and draw an amount of diluent into the syringe 104. By incrementally, pushing air into the vial 106 and drawing fluid into the syringe 104, over-pressurization of the vial 106 is minimized.

Similarly, the second limit marker 328 indicates a fill threshold and the third limit marker 330 indicates an empty threshold. The second limit marker 328 and the third limit marker 330 are configured to communicate with the actuator system 300 to stop moving or driving threaded screw 310 in response to the protrusion engaging with one of the second limit marker 328 or the third limit marker 330. According to this embodiment, the third limit marker 330 corresponds to the first position and the second limit marker 328 corresponds to the second position of the plunger 114.

The actuator system 300 further includes a gripper 332. The gripper 332 includes a gripper clamp 334 and a gripper rod 336. A first end of the gripper rod 336 is coupled to the frame 302 or the actuator plate 304 between threaded screw 310 and the guide rod 314. The gripper clamp 334 is coupled to the gripper rod 336 at a second end opposite the first end. The gripper clamp 334 extends through the casing 120 and is configured to receive the syringe barrel 108. The gripper clamp 334 is configured to couple the syringe barrel 108 (e.g., of a 50 mL syringe) to the actuator system 300 in a stationary position.

As shown in FIGS. 3-5, the device 102 further includes an electrical system such as a circuit board 402. The circuit board 402 is configured to communicably couple each of the limit markers 326, 328, 330 and the motor 306 of the actuator system 300.

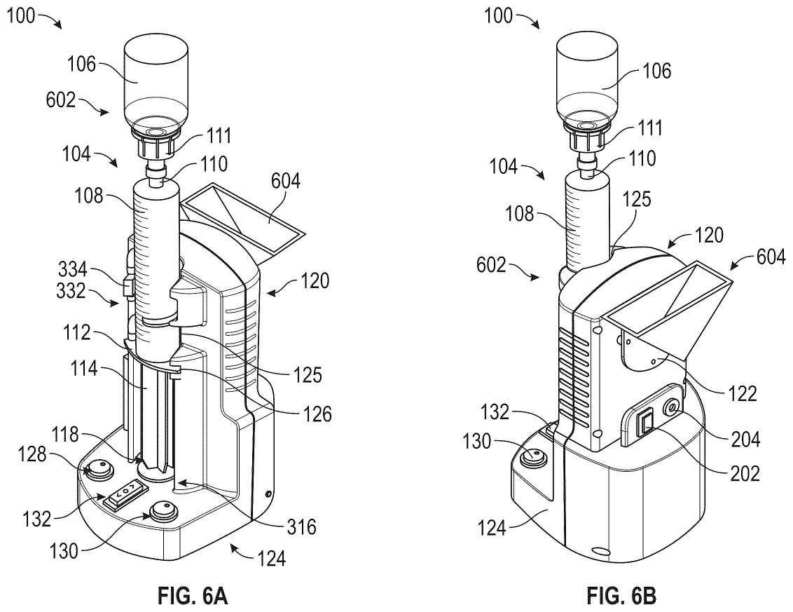

Now referring to FIGS. 6A and 6B, is the device 602 according to another embodiment. As shown in FIGS. 6A and 6B, the device 102 includes a cassette holder 604. The cassette holder 604 is coupled to a back side of the body portion 122 of the casing 120. The cassette holder 604 is configured to receive a cassette such that the cassette holder 604 can hold or secure the cassette to the device 102 when providing a fluid from the syringe 104 to the cassette.

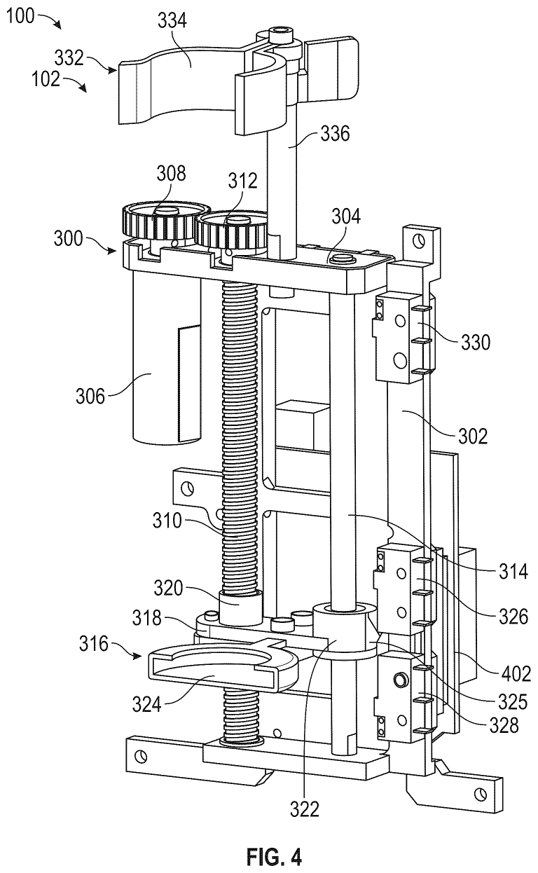

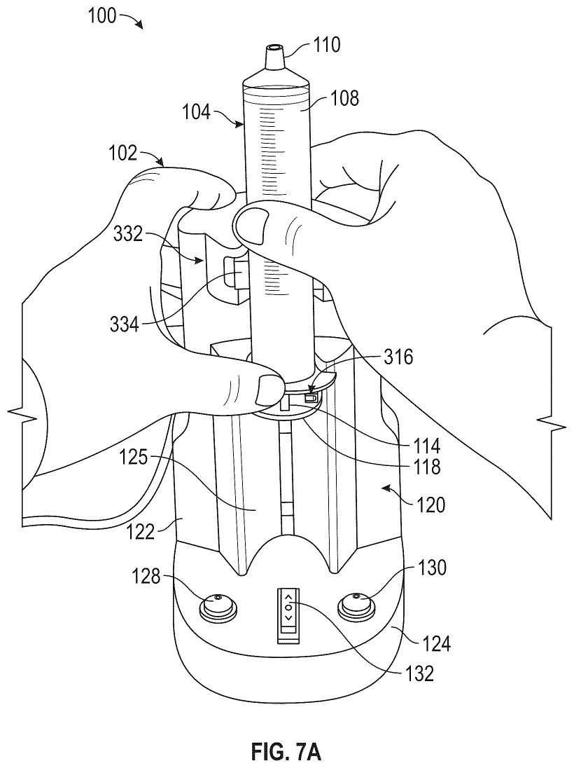

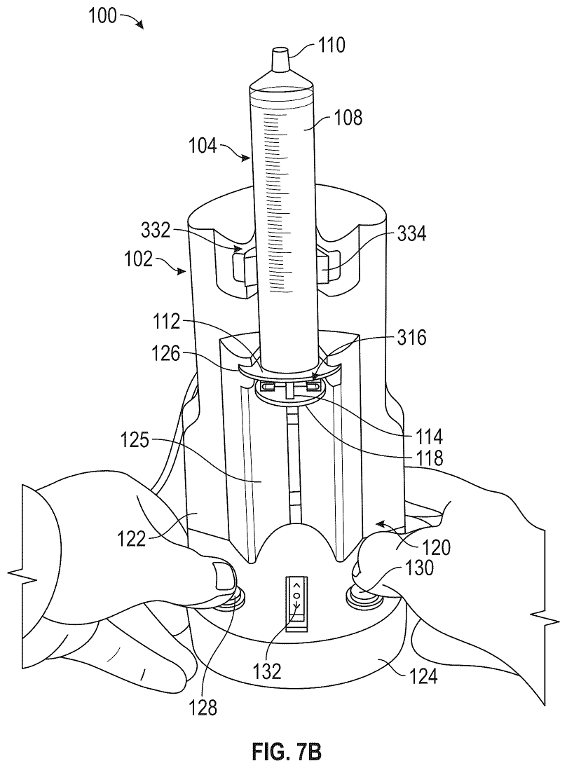

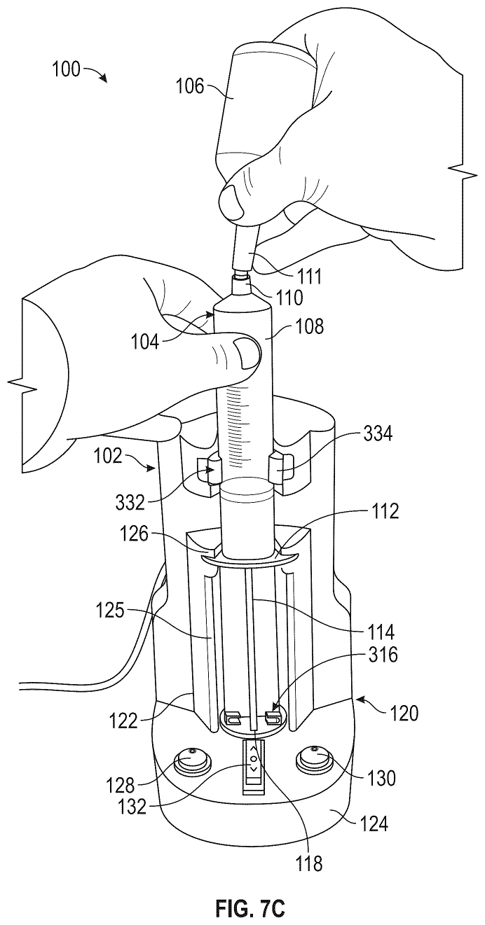

Now referring to FIGS. 7A-10 is a method for using the system 100. As shown in FIG. 7A, the syringe 104 is coupled to the device 102. For example, a user slides the body flange 112 into the body receptacle 126, slides the plunger base 118 into the slot 324, and presses the syringe barrel 108 into the gripper clamp 334. As shown in FIG. 7A, the user slides the body flange 112 into the body receptacle 126, slides the plunger base 118 into the slot 324 of the plunger holder 316, and presses the syringe barrel 108 into the gripper clamp 334 such that at least a portion of the syringe barrel 108 is positioned with the cylindrical cavity 125 (see, e.g., FIGS. 3-4). As such, the syringe 104 is vertically oriented with the nozzle 110 for receiving a fluid being accessible and positioned towards a top of the device 102. In some embodiments, the user engages the power switch 204 to provide electrical power to the device 102.

Then in FIG. 7B, the user engages each of the first button 128 and the second button 130. For example, the user engages, or presses and holds, each of the first button 128 and the second button 130. As described generally above, in response to or as a result of engaging each of the first button 128 and the second button 130, the circuit board 402 signals or controls the motor 306 to rotate first gear 308 which engages with the second gear 312 causing the threaded screw 310 to rotate. The threads of the first ring 320 engages with the threaded screw 310 such that the rotation of the threaded screw 310 causes the plunger holder 316 downward (e.g., from the first position to the second position, in a first direction, etc.) drawing or pulling air into the syringe 104. For example, the circuit board 402 controls the motor 306 to cause the plunger holder 316 to move downward until the protrusion 325 of the plunger holder 316 engages with or comes into contact with one of the limits markers 326, 328, 330. Specifically, the protrusion 325 engages with the limit marker 328 and the limit marker 328 provides a signal to the circuit board 402 in response to engagement with the protrusion 325. According to this embodiment, the limit marker 328 corresponds to a volume of about 50 mL. Thus, by moving the plunger holder 316 including the plunger base 118 downwards until the protrusion 325 engages with the limit marker 328, the syringe barrel 108 draws in about 50 mL of air.

As shown in FIG. 7C, the vial 106 is fluidly coupled to the nozzle 110 of the syringe 104. The vial 106 is orientated such that the vial 106 is vertically aligned with the syringe 104. As shown in FIG. 7C, the user flips the vial 106 such that the vial port is punctured, via the adapter 111, and fluidly coupled to the syringe 104.

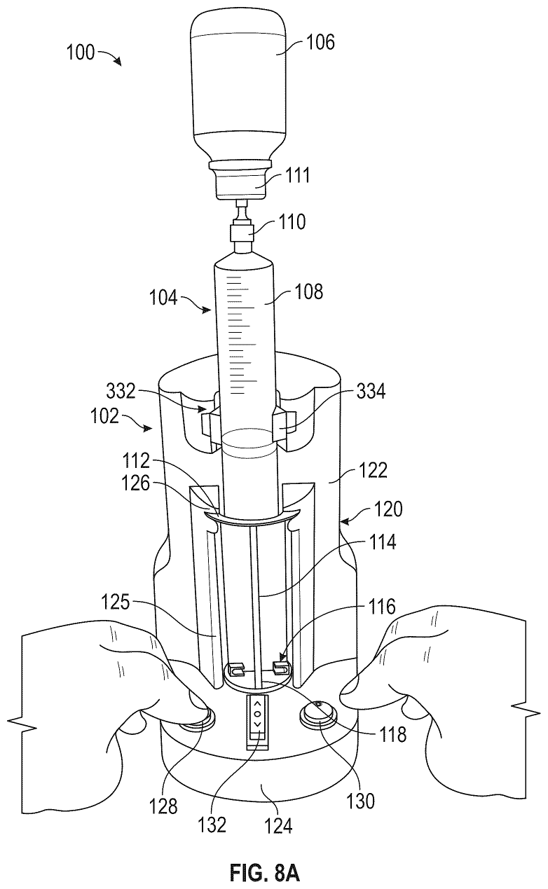

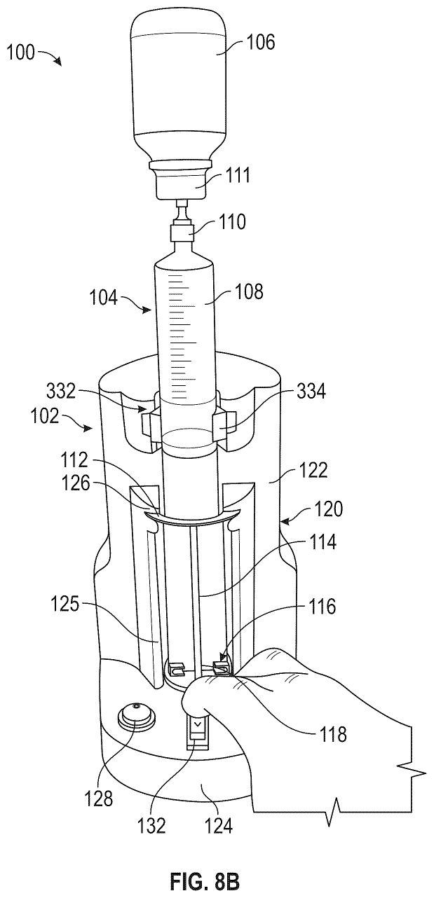

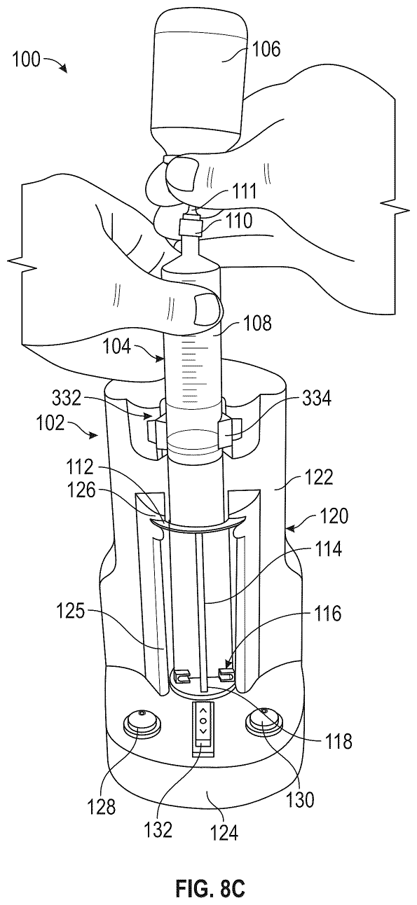

As shown in FIG. 8A, the user then engages or presses the first button 128. In response to the depression of first button 128, the circuit board 402 controls the motor 306 to rotate the first gear 308 (e.g., in a direction opposite the direction rotated when engaging each of the first button 128 and the second button 130 as shown in FIG. 7B, etc.) that engages with the second gear 312 coupled to the threaded screw 310 moving the plunger holder 316 including the plunger base 118 upward from the second position to the third position (e.g., in a second direction opposite the first direction, etc.) causing air that was drawn into the syringe barrel 108 to be pressed into the vial 106.

Pressing air into the vial 106 increases the pressure within the vial 106 facilitating the extraction of or drawing out of fluid from within the vial 106. The air pressed into the vial 106 also minimizes the formation of a vacuum within the vial 106. Vacuum formation can make increase resistance when drawing fluid of the vial 106.

Once the air in the syringe barrel 108 is pressed into the vial 106 and the protrusion 325 engages with the limit marker 326, the circuit board 402 receives a signal from the limit marker 326 and the circuit board 402 is configured to, automatically or in response to the protrusion 325 engaging with the limit marker 328 (e.g., a filling limit marker, etc.), cause the motor 306 to rotate the first gear 308 engaging with the second gear 312 coupled to the threaded screw 310 to move the plunger holder 316 including the plunger base 118 downward from the third position to the second position (e.g., in the first direction, etc.) drawing the fluid into the syringe barrel 108 until the protrusion 325 engages with the limit marker 328. For example, moving the plunger holder 316 including the plunger base 118 from the second position (e.g., from limit marker 328, etc.) to the third position (e.g., limit marker 326, etc.) and from the third position back to the second position, can cause an amount of air (e.g., an amount less than the entire volume of the barrel 108, etc.) to be pressed into the vial and an amount of fluid (e.g., less than the total volume of the barrel 108, about 5 mL, about 10 mL, etc.) to be repeatedly drawn into the barrel 108.

Once the protrusion 325 engages with the limit marker 328 (e.g., corresponding to the second position, etc.), the limit marker 328 provides a signal to the circuit board 402 and the circuit board 402 controls the motor 306 to repeatedly rotate the first gear 308 engaged with the second gear 312 coupled to the threaded screw 310 moving the plunger holder 316 including the plunger base 118 upward from the second position to the third position (e.g., in the second direction, etc.) and downward from the third position back to the second position (e.g., in the first direction, etc.) until the barrel 108 is filled to the desired amount (e.g., filled with 50 mL diluent, filled with 47 mL of diluent, etc.). In other embodiments, the user may manually hold the first button 128 until the air is pressed out of the syringe barrel 108 and then press and/or hold the first button 128 again until the syringe barrel 108 is filled with the desired amount of fluid. In other embodiments the user may press and/or toggle the third button 132 to press air into the vial 106 and draw fluid in the barrel 108. In some embodiments, the actuator system 300 is configured to stop rotating threaded screw 310 (e.g., stop moving the plunger holder 316 including the plunger base 118, etc.) in response to a user input, such as a press of the first button 128 or the second button 130.

As shown in FIG. 8B, the user then engages or toggles and/or presses the third button 132 to adjust an amount of fluid within the syringe barrel 108. For example, if 50 mL of fluid is drawn into the syringe barrel 108 and only 47 mL of fluid is desired, then the user can toggle the third button 132 causing an amount of fluid to be drawn in or pressed out of the syringe barrel 108 until the desired amount of 47 mL is reached. The third button 132 is configured to, when engaged, cause the actuator system 300 to move the plunger holder 316 including the plunger base 118 in either the first direction (e.g., toward the second position or the base portion 124, etc.) or the second direction (e.g., toward the first position, a direction away from the base portion 124, etc.).

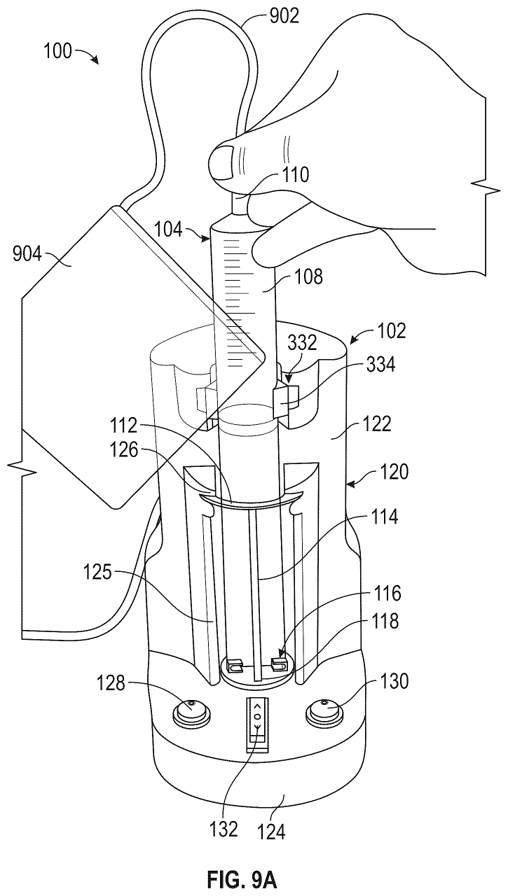

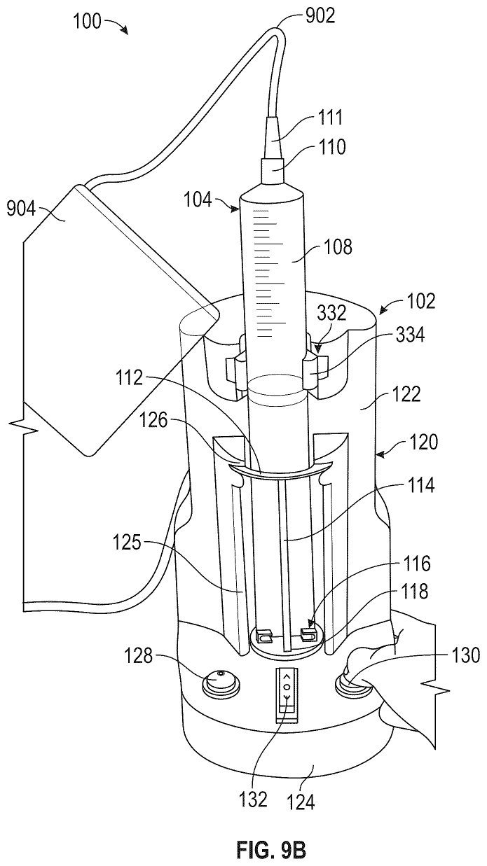

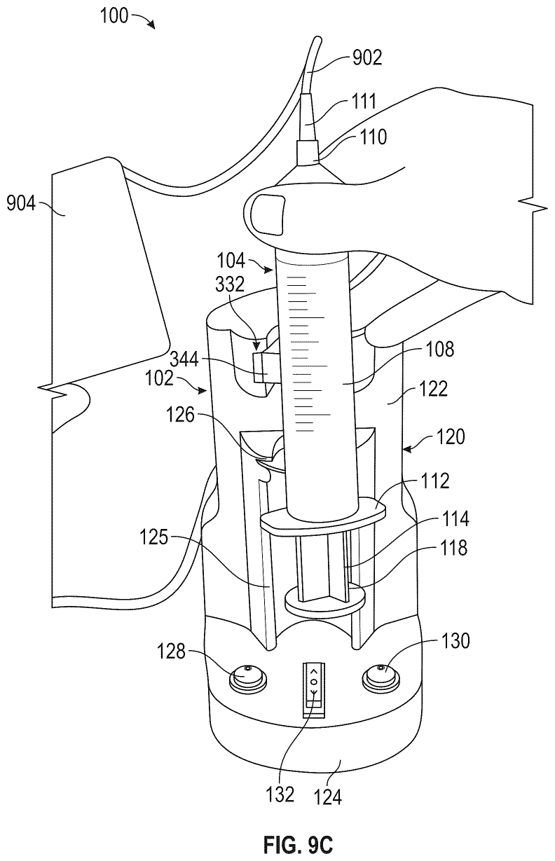

As shown in FIG. 8C, once the syringe barrel 108 is filled to the desired amount (e.g., 47 mL, 50 mL, etc.), the user decouples, or releases, the vial 106 from the syringe 104. As shown in FIGS. 9A-9C, the system 100 is configured to fluidly couple to a CADD cartridge or cassette 902 via tubing 904. As shown in FIG. 9A, the user couples the tubing 904 to the syringe 104 fluidly coupling the syringe 104 to the cassette 902. In other embodiments, the system 100 is configured to couple to any reservoirs with a Luer lock adapter, such as other types of tubing, an IV bag, or other syringes.

As shown in FIG. 9B, by engaging or pressing (e.g., by the user, etc.) the second button 130 the actuator system 300 is configured to rotate threaded screw 310 moving the plunger holder 316 including the plunger base 118 upward or vertically upward, from the second position (corresponding to the limit marker 328) to the first position (corresponding to the limit marker 330) (e.g., in the second direction, etc.) causing the fluid to be pressed out of or expelled from the syringe barrel 108 and into the cassette 902. For example, the motor 306 is configured to rotate the first gear 308 that engages with the second gear 312 coupled to the threaded screw 310 moving the plunger holder 316 including the plunger base 118 upward until the protrusion 325 engages with or makes contact with the limit marker 330. In some embodiments, the user may engage, press, or toggle the third button 132 causing excess fluid in the syringe barrel 108 to be pressed back into the vial 106 or cause additional fluid to be drawn into the syringe barrel 108. As shown in FIG. 9C, once the cassette 902 is filled, the user can then decouple or remove the syringe 104 from the device 102 while keeping the syringe 104 fluidly coupled to the cassette 902 via the tubing 904.

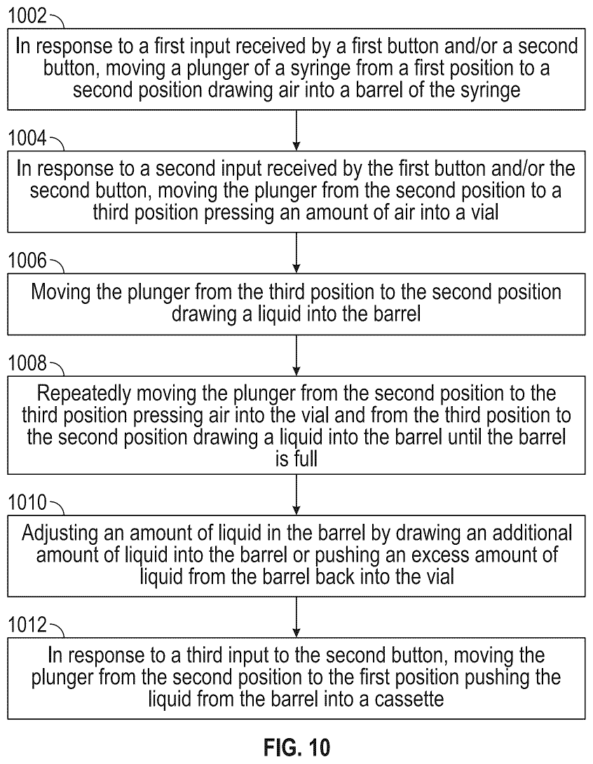

FIG. 10 is a flow chart illustrating the method as described and shown in FIGS. 7A-9C. The method 1000 includes providing inputs, such as pressing or engaging buttons 128, 130, 132 of the device 102 to control the actuator system 300. First, the user provides a first input, such as pressing the first button 128 and/or pressing the second button 130 (simultaneously or at the same time), to the device. For example, at step 1002, in response to the first input received by the first button 128 and or the second button 130 of the device 102, the circuit board 402 controls the motor 306 to move the plunger 114 of a first syringe 104 from a first position (corresponding to limit marker 330) to a second position (corresponding to limit marker 328) drawing air into a barrel of the syringe. According to this embodiment, the first button 128 and the second button 130 are pressed simultaneously to provide the first input. For example, the circuit board 402 determines the first input occurs in response to both the first button 128 and the second button 130 being depressed at the same time.

A second input is then provided to the device. For example, at step 1004, in response to the second input being received by the first button 128 and/or the second button 130, the circuit board 402 causes the motor 306 to move the plunger 114 of the syringe 104 from the second position to the third position (e.g., corresponding to limit marker 326, an intermediate position, a position between the first and second position, etc.) pushing air (an amount less than the total volume of the barrel 108) into a first vial 106. According to this embodiment, the first button 128 to provide the second input. For example, the circuit board 402 determines the second input occurs in response to both the first button 128 and the second button 130 being depressed at the same time.

Then, the plunger 114 is moved from the third position (e.g., an intermediate position, etc.) to the second position by the actuating system 300, drawing a liquid medication into the barrel 108 of the syringe 104. For example, at step 1006 the actuator system 300 moves the plunger 114 from the third position to the second position drawing a diluent from the first vial 106 into the barrel 108 of the syringe 104 occurs. According to this embodiment, step 1006 can occur automatically after step 1004. In other embodiments, the user may control the device 102 by providing an input (e.g., pressing at least one of the first button 128 or the second button 130, etc.) for step 1006 to occur.

The actuator system 300 repeatedly or cyclically moves the plunger 114 from the second position to the third position pressing an amount of air into the vial and moving the plunger 114 from the third position back to the second position drawing liquid, or diluent, into the barrel 108 until the syringe barrel 108 is filled with a desired amount of liquid (e.g., 50 mL of diluent, 47 mL of diluent, etc.). For example, at step 1008, the actuator system 300 is configured to repeatedly move the plunger 114 from the second position to the third position pressing air into the vial and from the third position to the second position drawing a liquid into the barrel 108 until the barrel 108 is full or contains a desired amount of liquid.

Optionally, the user may adjust the amount of liquid, such as diluent, within the syringe barrel 108 of the syringe 104. For example, if too much diluent is drawn into the syringe barrel 108, the user may push a small amount (e.g., 1 mL, 2 mL, etc.) out of the syringe barrel 108 by pressing the third button 132. Similarly, if an additional amount of medication is needed with in the syringe barrel 108, the user may draw more diluent into the syringe barrel 108 by pressing the third button 132. For example, at step 1010, the actuator system 300 is configured to adjust an amount of liquid in the barrel 108 by drawing an additional amount of liquid into the barrel 108 or pushing an excess amount of liquid from the barrel 108 back into the vial 106. For example, the user may toggle the third button 132 (e.g., press a first portion such as a bottom portion, or press a second portion such as a top portion, etc.).

Then the medication in the barrel 108 of the syringe 104 is pressed into or expelled into the cassette 902. The actuator system 300 moved the plunger 114 from the second position to the first position causing the medication to be pressed out of the syringe barrel 108 and into the cassette 902. For example, at step 1012, in response to a third input to the second button 130, the actuator system 300 is configured to move, by the actuating system 300, the plunger 114 of the first syringe 104 from the second position to the first position pushing the liquid, such as a diluent, into a cassette 902.

Multiple syringes 104 may be used with the device 102. For example, a 50 ml syringe, may be used for extracting the diluent from the vial 106, but in other embodiments, syringes 104 comprising a volume of more or less than 50 mL may be used (e.g., 100 mL). Steps 1002-1012 may be repeated to fill a plurality of syringes 104. For example, the user may repeat steps 1002-1012 to fill two or three syringes 104 (e.g., 100 mL syringes, 50 mL syringes, etc.) of diluent and provide the diluent to the cassette 902 for mixing with a medication.

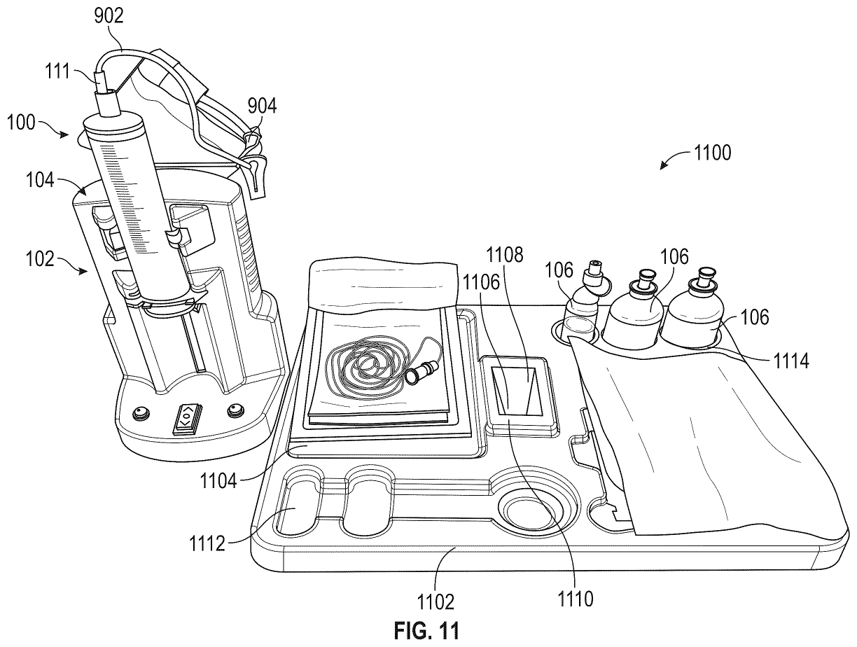

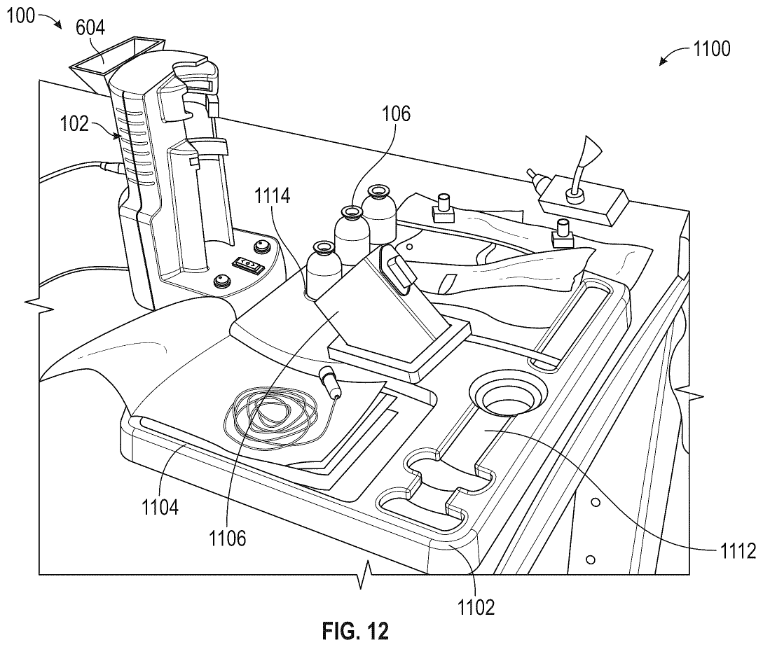

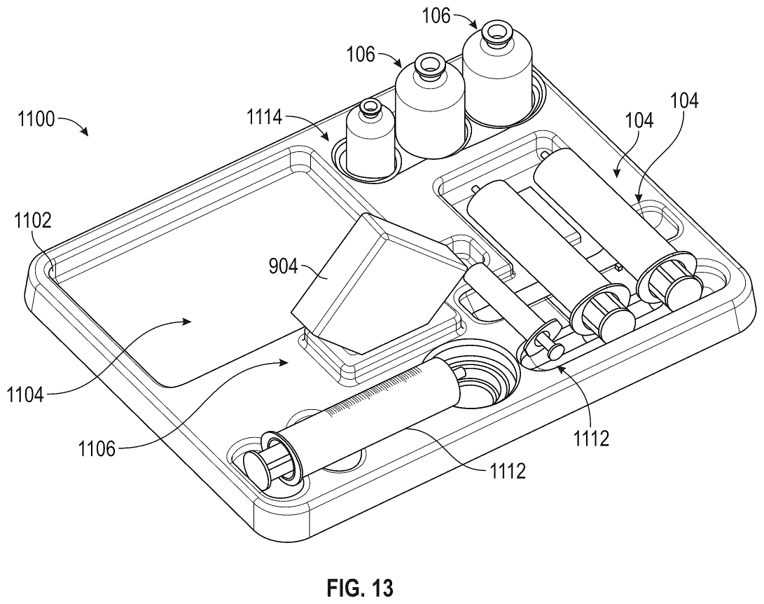

Now referring to FIGS. 11-14 are images of a kit 1100 including the system 100 and the device 102. The kit 1100 includes a tray 1102. The tray 1102 is clean and provides an area to place and prepare materials for assembling the system 100. For example, the tray 1102 is configured to hold vials 106 and syringes 104 to mitigate movement of the syringes 104 and/or spilling of the contents of the vials 106.

The tray 1102 includes a multi-purpose area 1104. The multi-purpose area 1104 is substantially rectangular and can be used to store various material such as tubing 904.

The tray 1102 also includes a cassette area 1106. The cassette area 1106 includes two angled sides 1108 and 1110. The angled sides 1108 and 1110 are configured to facilitate holding a cassette, such as cassette 902 in an upright position. For example, the sides 1108 and 1110 are configured to hold a filled cassette in place such that air within the cassette 902 moves towards the top of the cassette 902 for extraction and minimizes the risk of spilling the filled cassette.

The tray 1102 further includes syringe holders 1112 (e.g., three syringe holders 1112, four syringe holders 1112, etc.). The syringe holders 1112 are configured to store various syringes 104 of varying size. The tray 1102 also includes vial holders 1114. The vial holders 1114 are configured to store and or hold vials 106 to minimize spilling and maintain sterility of the ends of the vial 106 that engage with the syringe 104.

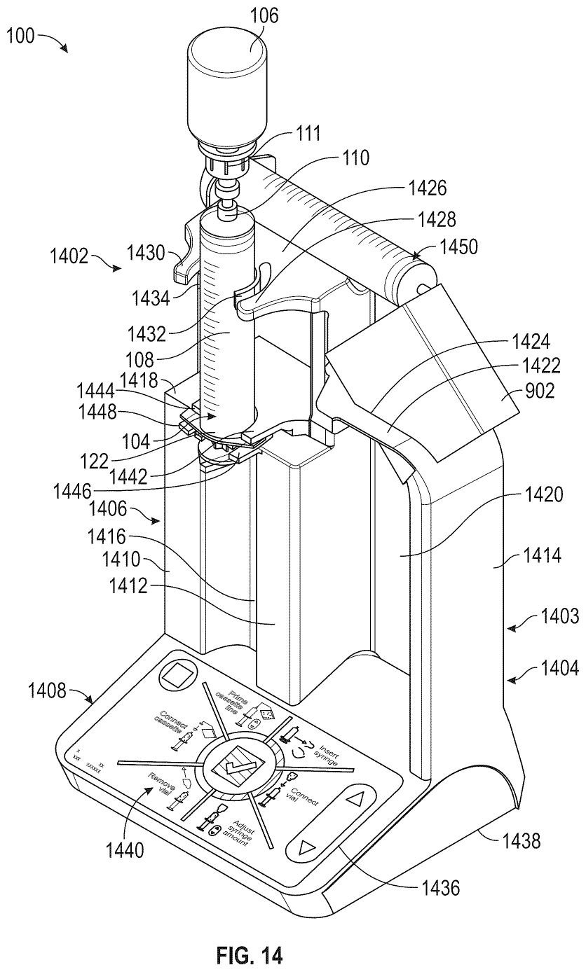

FIGS. 14-18 illustrate portions of the system 100, according to some embodiments, for filling a syringe with a liquid, such as a diluent, and providing the liquid into a cassette or other device for mixing fluids, such as diluents, buffers, and medications in preparation for delivery. The system includes a device 1402, the syringe 104, and a first fluid container, such as the vial 106.

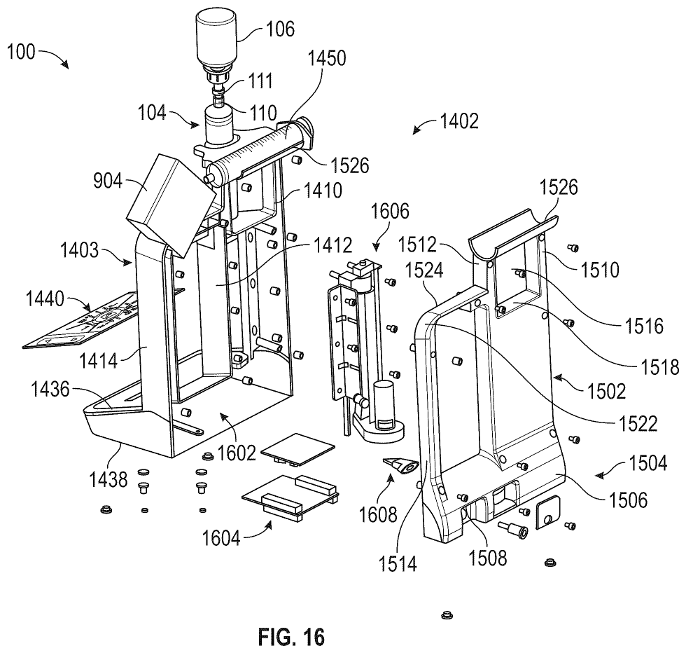

As shown in at least FIGS. 14 and 16, is a front portion 1403 of the device 1402 includes a casing 1404. According to this embodiment, the casing 120 is manufactured of or comprises a plastic material. The casing 1404 is configured to receive the syringe 104 for filing the syringe 104 with a comprising a diluent or a medication, such as treprostinil or a pharmaceutically acceptable salt or ester thereof.

The casing 1404 includes a body portion 1406 and a base portion 1408. The base portion 1408 extends outward from a lower end of the body portion 1406. The base portion 1408 is configured to be in contact with a surface. The base portion 1408 is configured to support and position the base portion 1408 in an upright or standing position.

The body portion 1406 includes a first front support 1410, a second front support 1412, and a third front support 1414. Each of the first front support 1410, the second front support 1412, and the third front support 1414 extend from the base portion 1408. For example, each of the first front support 1410, the second front support 1412, the third front support 1414 extend upward substantially perpendicular to the base portion 1408. The second front support 1412 is positioned a distance away from the first front support 1410 defining a first gap therebetween. The third front support 1414 is positioned a distance away from the second front support 1412 defining a second gap therebetween. The first front support 1410, the second front support 1412, and the third front support 1414 are substantially the same length.

The body portion 1406 also includes a fourth front support 1416. The fourth front support 1416 extends between the first front support 1410 and the second front support 1412. The first front support 1410, the second front support 1412, and the fourth front support 1416 define a syringe cavity therebetween. For example, each of the first front support 1410, the second front support 1412, and the fourth front support 1416 are configured to receive the syringe 104. When in an extended or filled position, the plunger 114 and the plunger base 118 of the syringe 104 extend between the first front support 1410 and the second front support 1412.

The body portion 1406 includes a flange support plate 1418. The flange support plate 1418 is coupled to an end with each of the first front support 1410, the second front support 1412, and the third front support 1414. The flange support plate 1418 has a thickness such that flange support plate 1418 defines a slot 1420 between the first front support 1410 and the second front support 1412 and the flange support plate 1418 for receiving the body flange the syringe 104 therebetween.

The third front support 1414 includes a front cassette support portion 1422. The front cassette support portion 1422 extenders from the third front support 1414 to the second front support 1412. For example, the front cassette support portion 1422 defines a curve or bend such that the front cassette support portion 1422 extends substantially perpendicular to each of the third front support 1414 and the second front support 1412. According to this embodiment, the front cassette support portion 1422 is positioned along a plane that is vertically offset from the flange support plate 1418. For example, the front cassette support portion 1422 is shown as being positioned along a plane higher than a plane of the flange support plate 1418.

The front cassette support portion 1422 defines an first portion of an opening 1424. The first portion of an opening 1424 is substantially rectangular in shape, but other shapes are possible (e.g., square, oval, circular, etc.). the first portion of an opening 1424 is configured to receive the cassette 902. For example, the edges of the first portion of an opening 1424 are configured to receive the cassette 902 and position the cassette 902 such that the cassette 902 is positioned in an upright position (e.g., in a diamond orientation, etc.) to facilitate viewing of the liquid or medicine within the cassette 902 during or after filling.

The body portion 1406 also includes a syringe body support plate 1426. The syringe body support plate 1426 extends substantially perpendicular to the first front support 1410 and the third front support 1414. The syringe body support plate 1426 extends along a horizontal plane positioned vertically offset (e.g., above the flange support plate 1418 and the front cassette support portion 1422, etc.).

The syringe body support plate 1426 includes a first distal arm 1428, a second distal arm 1430, a first proximal arm 1432, and a second proximal arm 1434. Each of the first distal arm 1428, the second distal arm 1430, the first proximal arm 1432, and the second proximal arm 1434 extend outward from the syringe body support plate 1426. The first proximal arm 1432 is positioned adjacent to and proximal to the first distal arm 1428 defining a gap therebetween, and the second proximal arm 1434 is positioned adjacent to and proximal to the second distal arm 1430 defining a gap therebetween. The gap defined between each of the first distal arm 1428 and the first proximal arm 1432 and the second distal arm 1430 and the second proximal arm 1434 facilitate movement of the first proximal arm 1432 and the second proximal arm 1434. The first proximal arm 1432 and the second proximal arm 1434 are configured to receive the syringe barrel 108 of the syringe 104, for example spaced and shaped such that the the syringe barrel 108 can be press fit between the first proximal arm 1432 and the second proximal arm 1434. The gap defined between each of the first distal arm 1428 and the first proximal arm 1432 and the second distal arm 1430 and the second proximal arm 1434 facilitate the movement or flexion of the first proximal arm 1432 and the second proximal arm 1434 in a direction towards each of the first distal arm 1428 and the second distal arm 1430 (e.g., when the syringe barrel 108 is being press-fit between the first proximal arm 1432 and the second proximal arm 1434, etc.). Collectively, the first proximal arm 1432 and the second proximal arm 1434 can be referred to as a “gripper” or device that grips or holds the syringe barrel 108 of the syringe 104.

The base portion 1408 defines a front face 1436 and a bottom face 1438. The bottom face 1438 is configured to be in contact with a surface, such as a table. The front face 1436 extends at an angle relative to the bottom face 1438. For example, the front face 1436 extends at an angle in a range between 30 degrees and 75 degrees (e.g., 45 degrees, etc.). The position of the front face 1436 facilitates viewing of the front face 1436 when using the device 1402 to fill the syringe 104.

The base portion 1408 includes a display 1440. The display 1440 is positioned on the front face 1436. The display 1440 is configured to receive inputs from a user as described in more detail herein.

As shown in FIG. 14, the first front support 1410 defines a first protrusion 1442 and a second protrusion 1444. Each of the first protrusion 1442 and the second protrusion 1444 extends substantially perpendicular outwards from each of the first front support 1410 and the second front support 1412. The first protrusion 1442 and the second protrusion 1444 are configured to engage or contact the body flange 112. In an empty or unfilled positioned, the plunger base 118 is positioned adjacent to each of the first protrusion 1442 and the second protrusion 1444. As the syringe 104 is filled, the plunger base 118 is pulled or moved in a direction away from each of the first protrusion 1442 and the second protrusion 1444 towards the base portion 1408.

The flange support plate 1418 includes a third protrusion 1446 and a fourth protrusion 1448. The third protrusion 1446 is positioned a distance away from and adjacent to the first protrusion 1442 and the fourth protrusion 1448 is positioned a distance away from and adjacent to the second protrusion 1444. The body flange 112 is positioned in the slot 1420 adjacent to each of the first protrusion 1442, the second protrusion 1444, the third protrusion 1446, and the fourth protrusion 1448. Each of the first protrusion 1442, the second protrusion 1444, the third protrusion 1446, and the fourth protrusion 1448 are configured to engage with or contact the body flange 112 to facilitate holding or keeping the syringe 104 in a stationary position while filling.

According to this embodiment, the system 100 includes a second syringe 1450. The second syringe 1450 is configured to be stored or positioned on the device 1402 as described in more detail herein.

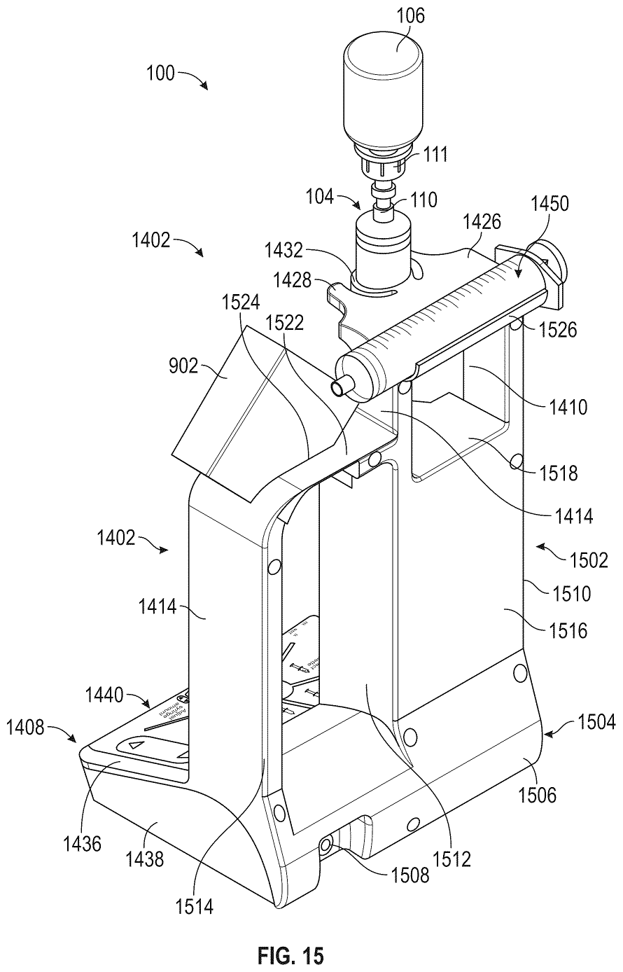

As shown in FIGS. 15 and 16, is a back side 1500 of the device 1402. The device 1402 includes a back body portion 1502. The back body portion 1502 is configured to be coupled to the front portion 1403. In some embodiments, the back body portion 1502 is removably coupled to the front portion 1403. For example, the back body portion 1502 may be removable coupled to the front portion 1403 such that the back body portion 1502 can be removed for servicing the device 1402.

The back body portion 1502 includes a base back portion 1504. The base back portion 1504 is coupled to the base portion 1408. The base back portion 1504 also includes a back face 1506. The back face 1506 defines a port 1508. The port 1508 is configured to receive power (e.g., from a power cord, from a power bank, etc.).

The back body portion 1502 also includes a first back support 1514. The first back support 1514 extends from the back face 1506 to the front cassette support portion 1422. The first back support 1514 is configured to be aligned with and coupled to the third front support 1414.

The back body portion 1502 also includes a second back support 1516. The second back support 1516 is extends from the back face 1506. The second back support 1516 is configured to be coupled to each of first front support 1410, the second front support 1412, and the fourth front support 1416.

The second back support 1516 includes an end surface 1518. The end surface 1518 of the second back support 1516 is configured to align or abut the flange support plate 1418.

The back body portion 1502 also includes a back cassette support portion 1522. The back cassette support portion 1522 extends from the first back support 1514 to the second back support 1516. For example, the back cassette support portion 1522 includes a bend or curve and extends substantially perpendicular to the first back support 1514. The back cassette support portion 1522 aligns with or abuts at least a portion of the front cassette support portion 1422.

The back cassette support portion 1522 defines a second portion of the opening 1524. The second portion of the opening 1524 cooperates with the opening 1424 to define the rectangular shape opening. The edges of the first portion of the opening 1424 and the second portion of the opening 1524 are configured to contact the cassette 902.

The back body portion 1502 also includes a syringe holder 1526. The syringe holder 1526 is coupled to the second back support 1516. The syringe holder 1526 is curved or substantially U-shaped. The syringe holder 1526 is configured to receive the second syringe 1450. For example, the syringe holder 1526 stores an empty or unfilled second syringe 1450 before being filled.

As shown in FIG. 16, the front portion 1403 and the back body portion 1502 cooperate to define a cavity 1602. The cavity 1602 is configured to house or store components of the device 1402 as described herein.

The device 1402 includes electronics 1604. The electronics 1604 is positioned within the cavity 1602. The electronics 1604 is communicably coupled to the display 1440. The electronics 1604 is configured to receive an input from the display 1440 and provide a signal to portions of the device 1402 based on the received input.

The device 1402 includes an actuator system 1606. The actuator system 1606 is positioned within the cavity 1602. The actuator system 1606 is configured to move or actuate the plunger base 118 of the syringe 104 causing the syringe 104 to draw fluid or medicine into the syringe barrel 108 of the syringe 104. The actuator system 1606 can be controlled by the electronics, to provide the movements described herein.

The device 1402 is also shown as including a charge port 1608. The charge port 1608 is configured to allow power (electricity) to be communicated from an external power source to the electronics 1604 (e.g., via a power cord, USB cable, USB-C cable, etc.). The charge port 1608 may be connected to a power source during use of the device 1402 and/or the device 1402 may include one or more rechargeable batteries which can be charged via the charge port 1608 (such that the device 1402 can then be powered by stored energy from the one or more rechargeable batteries).

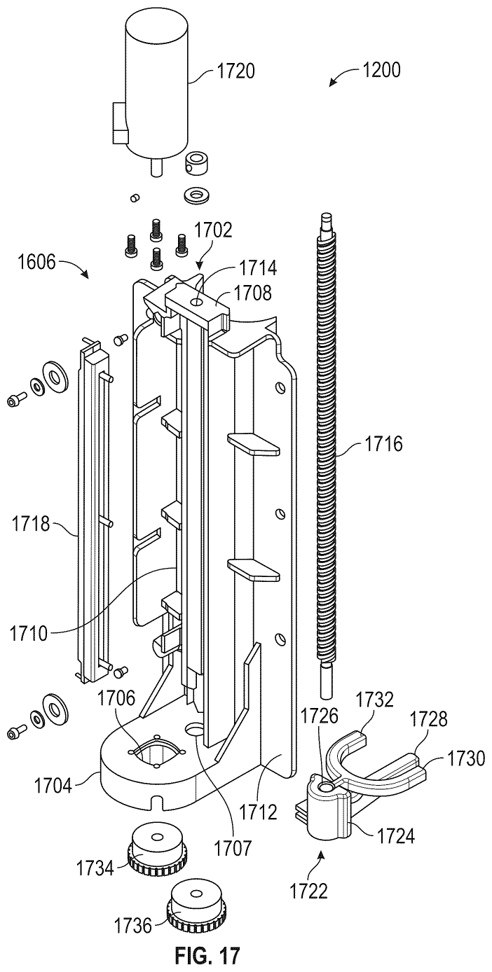

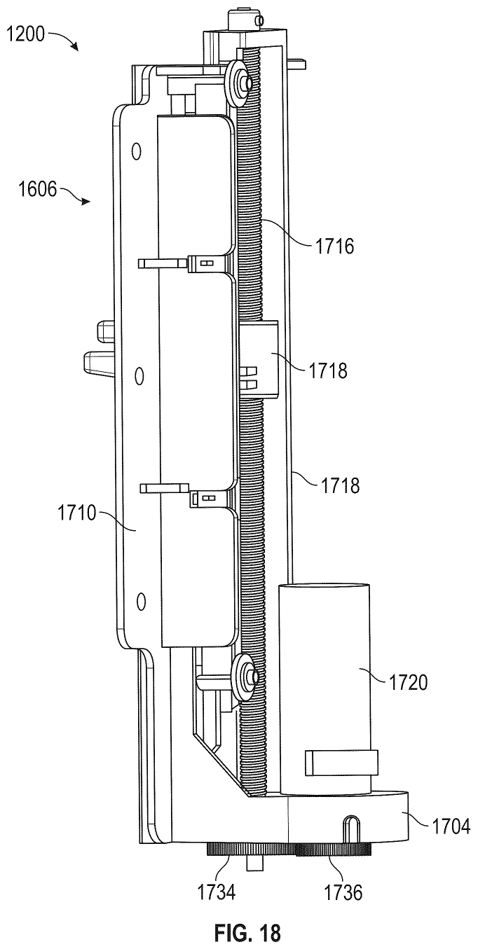

FIGS. 17 and 18 illustrate the actuator system 1606. The actuator system 1606 includes a frame 1702. The frame 1702 is configured to couple and/or position the actuating system within the casing 120. According to some embodiments, the frame 1702 is made of a metal or a hardened or rigid plastic.

The frame 1702 includes a frame base 1704. The frame base 1704 is positioned within the cavity 1602 of the base portion 1408 of the device 1402. The frame base 1704 is configured to support various components of the actuator system 1606. The frame base 1704 is substantially similar to the frame 302.

The frame base 1704 defines a first base aperture 1706. The first base aperture 1706 is configured to receive components of the actuator system 1606 as described in more detail herein. For example, the first base aperture 1706 is configured to receive, and couple, a motor (e.g., a motor 1720, etc.) to the frame 1702.

The frame base 1704 also defines a second base aperture 1707. According to this embodiment, the second base aperture 1707 is smaller than the first base aperture 1706. The second base aperture 1707. The second base aperture 1707 is configured to receive a component of the device 1402. For example, the second base aperture 1707 is configured to receive, and couple, a portion of a threaded screw or threaded rod (e.g., a threaded rod 1716, etc.).

The frame 1702 also includes a frame support 1708. The frame support 1708 is coupled to and extends from the frame base 1704. The frame support 1708 extends vertically from the frame base 1704. For example the frame support 1708 extends substantially perpendicular to the frame base 1704. The frame support 1708 is configured to support various components of the actuator system 1606 as described in more detail herein.

The frame 1702 also includes bracket portions 1710, 1712. The bracket portions 1710, 1712 extend laterally outward from the frame support 1708. The bracket portions 1710, 1712 are configured to receive fasteners (e.g., screws, etc.) to couple or secure the frame 1702 to one of the front portion 1403 or the back body portion 1502 of the casing 1404.

The frame support 1708 defines a first support aperture 1714. The first support aperture 1714 is defined on a top surface (e.g., a surface distal to the frame base 1704, etc.). The first support aperture 1714 is configured to receive a portion of a component, such as the threaded rod 1716, as described in more detail herein.

The actuator system 1606 also includes the threaded rod 1716. The threaded rod 1716 is coupled to the frame 1702. For example, the threaded rod 1716 is received by each of the second base aperture 1707 and the first support aperture 1714. The threaded rod 1716 extends perpendicular to the frame base 1704 and parallel to the frame support 1708. The threaded rod 1716 is configured to rotate (under power of the motor 1720) causing a portion of the actuator system 1606 to move axially along the threaded rod 1716 causing the syringe 104 to draw in a fluid (e.g., a medicine, etc.) or discharge the fluid.

The actuator system 1606 also includes a linear position sensor 1718. The linear position sensor 1718 is configured to measure the position of the plunger member 1722 along the threaded rod 1716 as the threaded rod 1716, as caused by rotation of the threaded rod 1716 under power of the motor 1720 (as described below). The linear position sensor 1718 can provide a measured value of the position of the plunger member 1722 to the electronics 1604, enabling the electronics 1604 to control the motor 1720 based on the measured position of the plunger member 1722. In the example shown, the linear position sensor 1718 is configured to provide substantially continuous position measurements along the full range of motion of the plunger member 1722 (e.g., along substantially the full length of the threaded rod 1716).

The actuator system 1606 also includes a motor 1720. The motor 1720 is coupled to the frame base 1704. For example, a portion of the motor 1720 is configured to be received by and positioned through the first base aperture 1706. The first base aperture 1706 is configured to rotate the threaded rod 1716 to cause the syringe 104 to draw fluid into or expel or discharge fluid out of the syringe barrel 108 of the syringe 104.

The actuator system 1606 also includes a plunger member 1722. A first portion of the plunger member 1722 couped to the threaded rod 1716 and a second portion of the plunger member 1722 is configured to receive a portion of the syringe 104 as described in more detail herein. For example, the plunger member 1722 is configured to receive the plunger base 118 of the syringe 104 and drive the plunger base 118 in directions towards and away from the base portion 1408 causing the syringe 104 to fill with fluid or expel fluid from the syringe barrel 108 of the syringe 104.

The plunger member 1722 includes a member base 1724. The member base 1724 defines a member opening 1726. The member base 1724 is configured to be coupled to the threaded rod 1716. For example, the member base 1724 is configured to be threaded onto the threaded rod 1716. As the threaded rod 1716 rotates or is driven or actuated by the motor 1720, the member base 1724 moves along the threaded rod 1716. The plunger member 1722 is configured to extend from the within the frame 1702 outside of the casing 1404 to receive a portion of the syringe 104.

The plunger member 1722 also includes a plunger interface 1728, a first plunger interface arm 1730, and a second plunger interface arm 1732. Each of the plunger interface 1728, the first plunger interface arm 1730, and the second plunger interface arm 1732 extend in a direction outward from the member base 1724. When the plunger member 1722 receives the plunger base 118 of the syringe 104 such that the plunger base 118 is coupled to the plunger member 1722, the plunger interface 1728 is positioned below or in contact with a distal surface of the plunger base 118 and each of the second button 130 and the second plunger interface arm 1732 are in contact with a proximal surface of the plunger base 118 (e.g., the plunger interface 1728 is in contact with or configured to engage with a bottom surface of the plunger base 118 and the first plunger interface arm 1730 and the second plunger interface arm 1732 are in contact or configured to engage with a top surface of the plunger base 118 opposite the bottom surface, etc.). For example, as the plunger member 1722 moves (or is driven by the motor 1720) in a direction towards the base portion 1408, each of the first plunger interface arm 1730 and the second plunger interface arm 1732 are in contact with the proximal surface of the plunger base 118 pulling the plunger base 118 in a direction towards the base portion 1408 causing the syringe barrel 108 to fill with medicine. As the plunger member 1722 moves in a direction away from the base portion 1408 (e.g., towards the vial 106, etc.), the plunger interface 1728 is in contact with or engaged with the distal surface of the plunger base 118 pushing the 118 towards the syringe barrel 108 of the syringe 104 causing the medicine or other fluid to be pushed or expelled out of the syringe barrel 108 of the syringe 104. Collectively, the plunger interface 1728, the first plunger interface arm 1730 and the second plunger interface arm 1732 can be referred to as a “plunger holder” that is configured to drive or move the plunger base 118 of the syringe 104 between a plurality of positions.

The actuator system 1606 also includes a first gear 1734 and a second gear 1736. Each of the first gear 1734 and the second gear 1736 are positioned on a side of the frame base frame base 1704. For example, the motor 1720 is positioned on a first side of the frame base 1704 and a portion of the motor 1720 extends through the first base aperture 1706 and is coupled to the first gear 1734 such that the first gear 1734 is positioned on a second side of the frame base 1704. The second gear 1736 is positioned on the second side of the frame base 1704 and is coupled to the threaded rod 1716. The motor 1720 is configured to drive the first gear 1734 in a direction (e.g., clockwise or counterclockwise). The first gear 1734 has grooves or teeth that engage with groove or teeth of the second gear 1736. The teeth of the first gear 1734 are configured to engage with the teeth of the second gear 1736 causing the first gear 1734 to translate the rotational motion from the motor 1720 to the second gear 1736 causing the threaded rod 1716 to rotate driving the plunger member 1722 in a direction toward or away from the frame base 1704.

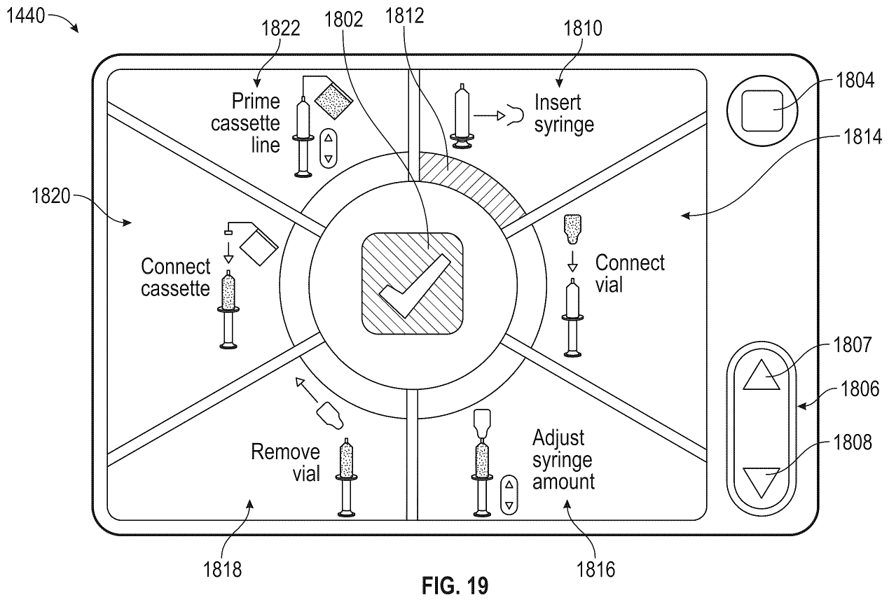

FIG. 19 illustrate the display 1440 of the device 1402 according to an embodiment. The display 1440 is a backlit display and user interface that is configured to receive inputs from the user while providing static, printed graphics which are selectively backlit to show and guide progress through a workflow using the device 1402. As shown, the display 1440 includes a center button 1802, in particular shown as a center button 1802 being square-like in shape and including a check mark (e.g., a green check mark). The center button 1802 is configured to illuminate or emit a green light to indicate that an input has been received or completed or that the center button 1802 is ready to be engaged to start the next step of the process of filling or expelling medicine from the syringe 104. In some embodiments, the center button 1802 is configured to be a “go” or “start” button. When engaged (e.g., pressed, etc.), the center button 1802 is configured to provide an input or signal to the actuator system 1606 causing the actuator system 1606 to proceed with or cause an action to occur (e.g., cause the motor 1720 to rotate the threaded rod 1716 moving the plunger member 1722 in a direction to draw fluid into or expel fluid from the syringe barrel 108 of the syringe 104, etc.).

The display 1440 also includes a stop button 1804. According to this embodiment, the stop button 1804 is circular and contains a square shape. The stop button 1804 is red or emits a red light (e.g., having one or more light-emitting diodes positioned behind a graphic of a stop button and operable to provide back-lighting such that the stop button 1804 illuminates). When engaged (e.g., pressed, etc.), the stop button 1804 is configured to provide an input or signal to the actuator system 1606 (e.g., via electronics 1604) causing the motor 1720 to stop operating (e.g., causing the plunger member 1722 to stop moving, causing the device 1402 to stop filling the syringe 104, to stop expelling medicine from the syringe 104, etc.). The stop button 1804 may be positioned in a corner of the display 1440 (e.g., the display 1440 in FIG. 14, etc.).

The display 1440 also includes an adjustment button 1806. The adjustment button 1806 includes a first portion 1807 and a second portion 1808. When engaged, the second base aperture 1707 is configured to cause the motor 1720 to drive the threaded rod 1716 in a first direction moving the plunger member 1722 towards the vial 106 causing fluid to be expelled or pushed out of the syringe barrel 108 of the syringe 104. When engaged, the second portion 1808 is configured to cause the motor 1720 to device the threaded rod 1716 in a second direction (e.g., opposite the first direction, etc.) moving the plunger member 1722 away from the vial 106 and towards the base portion 1408 drawing fluid into the syringe barrel 108 of the syringe 104 (e.g., filling the syringe barrel 108 with medicine). The adjustment button 1806 can be used as described above for the third button 132. The adjustment button 1806 may be positioned on the same side of the display 1440 as the stop button 1804 or positioned on an opposite side of the display 1440 than the stop button 1804 (e.g., as shown in the display 1440 of FIG. 14).

The display 1440 also includes a first display step 1810. The first display step 1810 is positioned radially outward of the center button 1802. The first display step 1810 includes an image and instructions. For example, the first display step 1810 includes an image of a syringe and includes the wording “insert syringe.”

The display 1440 includes an indicator ring 1812. The indicator ring 1812 is positioned around the center button 1802. The indicator ring 1812 includes segments corresponding to each of the display steps. For example, the indicator ring 1812 includes a segment corresponding to or associated with the first display step 1810. The indicator ring 1812 is configured to be illuminated (e.g., illuminated green, etc.) when the corresponding step is completed. For example, the indicator ring 1812 is configured to light up green once the actions described and/or shown in first display step 1810 are complete. In an embodiment, the first display step 1810 is configured to be illuminated indicating to or instructing the user to complete the action associated with the first display step 1810 (e.g., inserting the syringe 104, etc.) Once the action associated with the first display step 1810 is completed, the user may engage the center button 1802 to indicate that the action associated with the first display step 1810 is complete. In response to the action associated with the first display step 1810 being completed (e.g., the user engages the center button 1802 to indicate that it is complete, etc.), the segment of the indicator ring 1812 is illuminated. For example, the illumination of the segment of the indicator ring 1812 associated with eh first display step 1810 indicates or marks the action associated with the first display step 1810 as complete (e.g., the display 1440 is configured to track actions or tasks associated with filling and expelling fluid from the syringe 104, etc.).

The display 1440 also includes a second display step 1814, a third display step 1816, a fourth display step 1818, a fifth display step 1820, and a sixth display step 1822. Each of the second display step 1814, the third display step 1816, the fourth display step 1818, the fifth display step 1820, and the sixth display step 1822 display or illustrate another step or action associated with filling or expelling fluid into or from the syringe 104. For example, the second display step 1814 illustrates the syringe 104 and the vial 106 and includes the wording “Connect vial.” The third display step 1816 illustrates an image of the syringe 104, the vial 106 and the adjustment button 1806 and includes the wording “Adjust syringe amount.” The fourth display step 1818 illustrates the syringe 104 and the vial 106 being removed from the syringe 104 and includes the wording “Remove vial.” The fifth display step 1820 illustrates the syringe 104 and the cassette 902 being connected to the syringe 104 and includes the wording “Connect cassette.” The sixth display step 1822 illustrates the syringe 104, the cassette 902, and the adjustment button 1806 and includes the wording “Prime cassette line.”

The indicator ring 1812 includes segments associated with each of the second display step 1814, the third display step 1816, the fourth display step 1818, the fifth display step 1820, and the sixth display step 1822. After each action is complete, the user may engage the center button 1802 indicating the action is complete causing the associated segment of the indicator ring 1812 to be illuminated and causing the device 1402 to provide the subsequent step.

The first portion 1807 and the second portion 1808 of the adjustment button 1806 may be engaged at any time causing the threaded rod 1716 to rotate moving the plunger member 1722 drawing fluid into the syringe barrel 108 or pushing fluid out of the syringe barrel 108. In other embodiments, the adjustment button 1806 is configured to press fluid into or draw fluid out of the cassette 902.

The display 1440 can thereby guide a user through the steps to transfer fluid from a vial to a cassette using the device 1402, while accepting simple user inputs (e.g., press of the center button 1802) that cause the electronics 1604 to control the motor 1720 to move the plunger member 1722 in the appropriate movements for each step. Advantageously, the user can thus use the device 1402 to transfer fluid from a via to a cassette (via a syringe) without remembering details of the different steps required or different inputs to be provided to the device 1402, but rather by following the instructions in the display steps 1810, 1814, 1816, 1818, 1820, 1822 when the corresponding portion of the indicator ring 1812 illuminates (e.g., blinks, flashes) and pressing the center button 1802 to cause the device 1402 to automatically provide appropriate movements of a plunger of a syringe for each stage of a workflow for fluid transfer, according to the various teachings herein. The wording or instructions displayed in each of the display steps 1810, 1814, 1816, 1818, 1820, 1822 may vary and is not limited thereto.

As utilized herein with respect to numerical ranges, the terms “approximately,” “about,” “substantially,” and similar terms generally mean±/−10% of the disclosed values, unless specified otherwise. As utilized herein with respect to structural features (e.g., to describe shape, size, orientation, direction, relative position, etc.), the terms “approximately,” “about,” “substantially,” and similar terms are meant to cover minor variations in structure that may result from, for example, the manufacturing or assembly process and are intended to have a broad meaning in harmony with the common and accepted usage by those of ordinary skill in the art to which the subject matter of this disclosure pertains. Accordingly, these terms should be interpreted as indicating that insubstantial or inconsequential modifications or alterations of the subject matter described and claimed are considered to be within the scope of the disclosure as recited in the appended claims.

It should be noted that the term “exemplary” and variations thereof, as used herein to describe various embodiments, are intended to indicate that such embodiments are possible examples, representations, or illustrations of possible embodiments (and such terms are not intended to connote that such embodiments are necessarily extraordinary or superlative examples).

The term “coupled” and variations thereof, as used herein, means the joining of two members directly or indirectly to one another. Such joining may be stationary (e.g., permanent or fixed) or moveable (e.g., removable or releasable). Such joining may be achieved with the two members coupled directly to each other, with the two members coupled to each other using a separate intervening member and any additional intermediate members coupled with one another, or with the two members coupled to each other using an intervening member that is integrally formed as a single unitary body with one of the two members. If “coupled” or variations thereof are modified by an additional term (e.g., directly coupled), the generic definition of “coupled” provided above is modified by the plain language meaning of the additional term (e.g., “directly coupled” means the joining of two members without any separate intervening member), resulting in a narrower definition than the generic definition of “coupled” provided above. Such coupling may be mechanical, electrical, or fluidic.

References herein to the positions of elements (e.g., “top,” “bottom,” “above,” “below”) are merely used to describe the orientation of various elements in the figures. It should be noted that the orientation of various elements may differ according to other exemplary embodiments, and that such variations are intended to be encompassed by the present disclosure.

1. A device for transferring a fluid into and out of a syringe, the device comprising:
a frame;
a gripper coupled to the frame and configured to grip a barrel of the syringe; and
an actuator system coupled to the frame, the actuator system comprising:
a motor; and
a plunger holder configured to receive a plunger of the syringe when the gripper grips the barrel of the syringe,
wherein the motor is configured to drive the plunger holder between positions.

2. The device of claim 1, wherein the actuator system comprises:
a threaded screw coupled to the frame and comprising a first gear,
wherein the plunger holder is movably coupled to the threaded screw,
wherein the motor is coupled to the frame and a second gear, the second gear configured to engage with the first gear to rotate the threaded screw causing the plunger holder to move along the threaded screw.

3. The device of claim 2, wherein the plunger holder is movably coupled to the threaded screw and comprises: a first ring positioned around the threaded screw and comprising a first ring threading that engages with the threaded screw; and
a slot coupled configured to slidably receive a portion of the plunger.

4. The device of claim 1, further comprising one or more sensors configured to detect a position of the plunger holder, wherein the actuator system is operable to control the motor based on the detected position of the plunger holder.

5. The device of claim 1, wherein the actuator system is configured to control the motor to:
move the plunger holder from a first position to a second position to draw air into the barrel;
move the plunger holder from a second position to a third position to press air into a vial coupled to the syringe; and
move the plunger holder from the third position to draw fluid from the vial into the barrel.

6. The device of claim 1, wherein the actuator system is configured to repeatedly move, in response to a single press of a button by a user, the plunger holder up and down to progressively transfer fluid from a vial into the barrel while transferring air from the barrel into the vial.

7. The device of claim 6, further comprising additional buttons communicably coupled to the motor and configured to, when selected by the user, control the actuator system to selectively move the plunger in a first direction and a second direction opposite the first direction to adjust a volume of liquid in the barrel of the syringe.

8. The device of claim 1, wherein:
the device is configured to stand on a tabletop,
the gripper is configured to grip the syringe in a vertical orientation causing air within the barrel to move to an end of the barrel opposite the plunger.

9. A system comprising:
a syringe comprising a barrel and a plunger extending from a first end of the barrel;
a container configured to be coupled to the syringe; and
a device comprising:
a gripper configured to receive the barrel of the syringe in a vertical orientation; and
an actuator system comprising:
a frame;
a plunger holder configured to receive the plunger of the syringe when the gripper grips the barrel of the syringe, and
a motor coupled to the frame and configured to drive the plunger holder causing movement of the plunger of the syringe which transfers fluid between the container and the syringe.

10. The system of claim 9, wherein the device further comprises:
a first button communicatively coupled to the actuator system, wherein in response to the first button receiving an input, the actuator system is configured to control the motor repeatedly move, in response to a single press of a button by a user, the plunger holder up and down to progressively transfer fluid from the container into the barrel while transferring air from the barrel into the container.

11. The system of claim 10, further comprising an additional container configured to be selectively coupled to the syringe when the container is detached from the syringe, wherein in response to the first button receiving a second input, the actuator system is configured to drive the plunger holder to transfer fluid from the syringe into the additional container.

12. The system of claim 10, wherein:
the device comprises a switch communicatively coupled to the actuator system,
wherein when the switch is toggled, the switch is configured to activate the actuator system to move the plunger holder including the plunger in a first direction or a second direction to adjust a volume of liquid in the barrel of the syringe.

13. The system of claim 9, wherein:
the actuator system comprises a threaded screw coupled to the frame;
the motor is configured to rotate the threaded screw, and
the plunger holder is coupled to the threaded screw, the plunger holder comprising:
a first ring positioned around the threaded screw and comprising first ring threading, wherein the threaded screw comprises threading that engages with the first ring threading; and
a slot coupled configured to receive a portion of the plunger.

14. The system of claim 9, wherein the device further comprises a position sensor configured to detect position of the plunger holder, wherein the actuator system is configured to control the motor based on a signal from the position sensor.

15. The system of claim 9, further comprising a tray configured to hold at least the syringe, the container, and the device.

16. The system of claim 9, wherein a liquid in the syringe comprises diluent for mixing with treprostinil or a pharmaceutically salt or ester thereof.

17. A method of operating a device to transfer fluid from a vial to a cassette, the method comprising:
moving, by the device, a plunger of a syringe such that air is drawn into a barrel of the syringe;
moving, by the device, the plunger such that the air is pushed into the vial;
moving, by the device, the plunger such that fluid is drawn from the vial into the barrel of the syringe; and
detaching the vial from the syringe and attaching the cassette to the syringe; and
moving, by the device, the plunger to pushing the fluid from the syringe into the cassette.

18. The method of claim 17, further comprising:
in response to engagement of a portion of a button of the device, moving, by the device, the plunger to push an amount of fluid from the barrel back into the vial.

19. The method of claim 18, comprising repeatedly moving, by the device in response to a single user input, between positions to progressively push the air into the vial and draw the fluid from the vial.

20. The method of claim 19, wherein a mixture in the cassette of diluent with treprostinil or a pharmaceutically salt or ester thereof comprises a therapeutically effective amount of treprostinil or a pharmaceutically salt or ester thereof.
